(19) United States
(12) Patent Application Publication
(10) Pub. No.: US 2004/0014750 A1
(43) Pub. Date: Jan. 22, 2004

(54) Metal complexes and formulations of rifamycin analogues and uses thereof

(76) Inventors: Arthur F. Michaelis, Devon, PA (US); Hawkins V. Maulding, Mendham, NJ (US); Chalom Sayada, Luxembourg City, MA (US); Barry Eisenstein, Chestnut Hill, MA (US)

Correspondence Address: CLARK & ELBING LLP, 101 FEDERAL STREET, BOSTON, MA, 02110, US

(21) Appl. No.: 10/318,998

(22) Filed: Dec. 12, 2002

Related U.S. Application Data
(60) Provisional application No. 60/341,591, filed on Dec. 13, 2001.
(60) Provisional application No. 60/382,805, filed on May 23, 2002.
(60) Provisional application No. 60/385,532, filed on Jun. 3, 2002.
(60) Provisional application No. 60/406,873, filed on Aug. 29, 2002.
(60) Provisional application No. 60/412,958, filed on Sep. 23, 2002.

(51) Int. Cl. A61K 31/542; A61K 31/538

(52) U.S. Cl. 514/224.5; 514/229.5

(57) ABSTRACT

The invention features compositions that include rifamycin analogues formulated with metal salts, metal complexes of rifamycin analogues, and methods for treating disease using these compositions.

### CROSS-REFERENCE TO RELATED APPLICATIONS

The application claims benefit of U.S. Provisional Application Nos. 60/341,591, filed Dec. 13, 2001, 60/382,805, filed May 23, 2002, 60/385,532, filed Jun. 3, 2002, 60/406,873, filed Aug. 29, 2002, and 60/412,958, filed Sep. 23, 2002, which are hereby incorporated by reference.

### BACKGROUND OF THE INVENTION

The present invention relates to the field of antibacterial agents.

The use of antibiotics by humans can be seen as an evolutionary experiment of enormous magnitude, a window from which to view not-quite-natural selection operating in real time. Within 50 years, the number of species and strains of pathogenic and commensal bacteria resistant to antibiotics and the number of antibiotics to which they are resistant has increased virtually monotonically world-wide. As a result, infections that had been readily treatable by chemotherapy may no longer be so. It is clear that the evolution and spread of resistance can be attributed to the use and overuse of antibiotics. Increased resistance of bacterial infections to antibiotic treatment has been extensively documented and has now become a generally recognized medical problem, particularly with nosocomial infections. See, for example, Jones et al., Diagn. Microbiol. Infect. Dis. 31:379-388, 1998; Murray, Adv. Intern. Med. 42:339-367, 1997; and Nakae, Microbiologia 13:273-284, 1997.

Throughout the developed world there is public and governmental concern about the increasing prevalence of antimicrobial resistance to chemotherapy in bacteria that cause diseases in humans. Many pathogens exist for which there are few effective treatments, and the number of strains resistant to available drugs is continually increasing. New antimicrobial agents and improved methods are needed for the treatment and prevention of infections by such pathogens.

### SUMMARY OF THE INVENTION

The present invention features metal complexes and formulations comprising a rifamycin analogue and a metal. These can be used as therapeutics for treating or preventing a variety of bacterial infections.

In one aspect, the invention features a formulation including a metal salt and a rifamycin analogue of formula I:

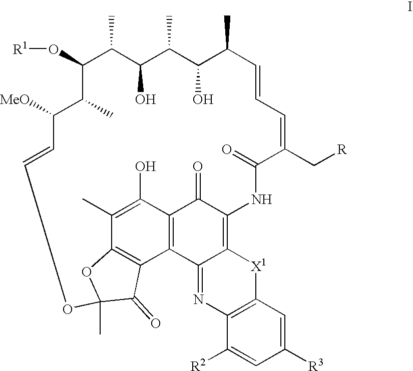

In formula I, X¹ represents an oxygen atom or a sulfur atom; R represents a hydrogen atom or hydroxyl group; R¹ represents acetyl or H, OH; R² represents a hydroxyl group or a sulfhydryl group; and R³ represents:

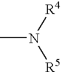

wherein each of R⁴ and R⁵ is, independently, an alkyl group having 1 to 7 carbon atoms, or R⁴ and R⁵ combine to form a 3-8 membered cyclic system,

or R³ represents a group expressed by the formula:

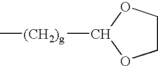

in which g represents an integer between 1 and 3,

or R³ represents a group expressed by the formula:

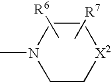

wherein each of R⁶ and R⁷ is, independently, a hydrogen atom or an alkyl group having 1 to 3 carbon atoms, X² represents an oxygen atom, a sulfur atom, or a carbonyl group,

or X² represents:

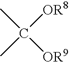

in which each of R⁸ and R⁹ is, independently, a hydrogen atom, or an alkyl group having 1 to 3 carbon atoms, or R⁸ and R⁹, in combination with each other, represent —(CH₂)k— in which k represents an integer between 1 and 4,

or X2 represents:

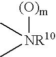

in which m represents 0 or 1, R¹⁰ represents a hydrogen atom, an alkyl group having 1 to 6 carbon atoms, or —(CH₂)ₙX³ in which n represents an integer between 1 and 4, and X³ represents an alkoxy group having 1 to 3 carbon atoms, a vinyl group, an ethynyl group,

or R¹⁰ represents:

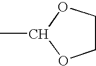

wherein the metal salt is added in an amount sufficient to reduce the minimum inhibitory concentration of the rifamycin analogue.

In related embodiments, the invention features a formulation comprising a metal salt and a rifamycin analogue described by formula II:

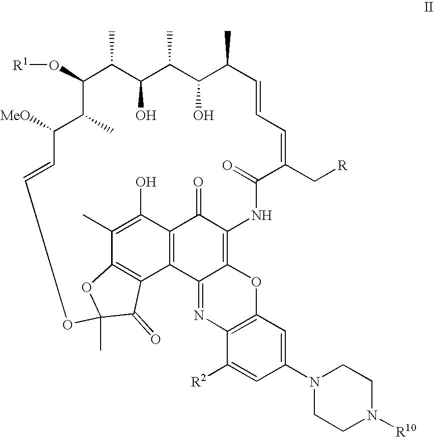

wherein R represents hydrogen or a hydroxyl group; R¹ represents hydrogen or an acetyl group; R² is hydroxyl or sulfhydryl; and R¹⁰ is selected from the group consisting of methyl, ethyl, iso-propyl, n-propyl, iso-butyl, (S)-sec-butyl, and (R)-sec-butyl.

In various embodiments, the metal salt comprises metals selected from the group consisting of Groups I (A, B), II (A, B), III (A, B), IV (A, B), V (A, B), VIA, VIIA, and VIII, and combinations thereof. Exemplary metals include aluminum, bismuth, chromium, cobalt, copper, gallium, germanium, gold, iridium, iron, manganese, nickel, palladium, platinum, rhenium, rhodium, ruthenium, selenium, silver, tin, titanium, vanadium, and zinc, and combinations thereof. In desirable embodiments the metal salt is iron.

Desirably, the mole ratio of metal to rifamycin analogue in the formulation falls within the range of 0.1 to 10.

In a related embodiment, the minimum inhibitory concentration of the rifamycin analogue formulated with a metal salt in a Chlamydia growth assay is less than 95%, 90%, 80%, 70%, 60%, 50%, 40%, 30%, 20%, 10%, 5%, 1%, or 0.1% of the minimum inhibitory concentration of the rifamycin analogue formulated without a metal salt.

In another aspect, the invention features a metal-rifamycin analogue complex comprising a metal salt and a rifamycin analogue of formulas I or II.

In various embodiments, the metal is selected from the group consisting of Groups I (A, B), II (A, B), III (A, B), IV (A, B), V (A, B), VIA, VIIA, VIII, and combinations thereof. Desirably, the metal is selected from the group consisting of aluminum, bismuth, chromium, cobalt, copper, gallium, germanium, gold, iridium, iron, manganese, nickel, palladium, platinum, rhenium, rhodium, ruthenium, selenium, silver, tin, titanium, vanadium, and zinc, and combinations thereof. For example, a complex can be formed by combining ferrous chloride with a rifamycin analogue of formulas I or II.

Where metals are available in multiple oxidation states, any one or a combination of salts of metals having differing oxidation states can be used in accordance with the present invention. For example, an iron-rifamycin analogue complex may include iron in an oxidation state of +4, +3, +2, +1, or combinations thereof.

The metal-rifamycin analogue complex may be described by formula III, wherein M is a metal, rifamycin analogue is an analogue of formulas I or II, and a and b each, independently, represent an integer from 1 to 10, inclusive.

Mₐ(rifamycin analogue)_{b} III

The invention features one or more of the following: a racemic mixture of two or more rifamycin analogues of the invention, two or more diastereomers of a rifamycin analogue of the invention, two or more metal-based structural isomers of formula III, two or more optical isomers of formula III, and two or more diastereomers of formulas III.

In a further embodiment, the invention features a mixture of two or more compounds of formula III.

The invention also features an aqueous solution comprising a metal-rifamycin analogue complex formed by the combination of a rifamycin analogue described by formulas I or II and the salt of a metal selected from the group consisting of Groups I (A, B), II (A, B), III (A, B), IV (A, B), V (A, B), VIA, VIIA, and VIII. The solution may be a pharmaceutical composition comprising one or more pharmaceutically acceptable salts, carriers or diluents.

The aqueous solution of a metal-rifamycin analogue complex can have a concentration of rifamycin analogue, including both complexed and uncomplexed forms, between 0.050 and 200,000 μg/mL. Desirably, the solution has a metal-rifamycin analogue complex concentration of between 0.050 and 100,000, 0.050 and 50,000, 0.10 and 200,000, 0.20 and 200,000, or 0.20 and 100,000 μg/mL.

In another aspect, the invention features a method of treating disease in a human by intravenous administration of an aqueous solution including a metal-rifamycin analogue complex in amounts effective to treat disease.

The metal-rifamycin analogue complex can be administered by intravenous infusion, wherein between 2 and 50 mg of the metal-rifamycin analogue complex is administered over a period of 4 to 24 hours. Desirably, between 4 and 40 mg, 4 25 and 30 mg, or 8 and 25 mg of the metal-rifamycin analogue complex is administered over a period of 4 to 24 hours, 8 to 24 hours, or 15 to 24 hours. Up to 2, 4, 6, 8, 10, 12, 14, 16, 18, 20, 22, 24, 26, 28, 30, 32, 34, 36, 38, 40, 42, 44, 46, 48, or 50 mg of the metal-rifamycin analogue complex is administered by intravenous infusion over a 2, 4, 5, 6, 7, 8, 9, 10, 12, 14, 20, 24, 48, or 72 hour period.

The metal-rifamycin analogue complex can be administered by intravenous bolus of between 2 and 25 mg of the metal-rifamycin analogue complex over a 10 to 60 minute period followed by a slow infusion of 0.1 to 2 mg, 0.5 to 2 mg, 0.5 to 1.5 mg, or 1 to 2 mg, per hour for up to 24 hours.

The intravenous administration of the metal-rifamycin analogue complex may be repeated daily or every other day, for a period of two to fourteen days. Desirably, the intravenous administration can be repeated every third day for a period of three to fifteen days.

The invention also features a method of treating disease in a human by intravenously administering a metal-rifamycin analogue complex at a rate that maintains a plasma concentration of the rifamycin analogue, including both complexed and uncomplexed forms, of between 4 and 80, 6 and 50, or 10 and 50 ng/mL for a period greater than 5, 8, 12, or 24 hours.

Desirably, the metal-rifamycin analogue complex is administered in a dosing schedule that maintains a plasma concentration of the rifamycin analogue, including both complexed and uncomplexed forms, of between 4 and 50, 6 and 60, or 6 and 40 ng/mL for a period greater than 24 hours.

The invention also features a method for treating or preventing the development of an atherosclerosis-associated disease in a patient by administering to the patient a metal complex or formulation of the invention in an amount effective to treat or prevent the development of the atherosclerosis-associated disease in the patient. The patient is typically diagnosed as having the atherosclerosis-associated disease (or being at increased risk of developing the disease) or as having macrophages or foam cells infected with C. pneumoniae prior to the administration of the metal complex or formulation.

The invention also features a method of reducing the level of C-reactive protein in a patient in need thereof by administering to the patient a metal complex or formulation of the invention in an amount effective to reduce the level of C-reactive protein in the patient. In one embodiment, the patient has not been diagnosed as having a bacterial infection. In another embodiment, the patient has been diagnosed as having macrophages or foam cells infected with C. pneumoniae.

The invention also features a method for reducing C. pneumoniae replication in macrophages or foam cells in a patient in need thereof by administering a metal complex or formulation of the invention to the patient in an amount effective to reduce C. pneumoniae replication in macrophages or foam cells in the patient.

The invention also features a method for treating a persistent C. pneumoniae infection in macrophages or foam cells in a patient by administering a metal complex or formulation of the invention to the patient in an amount effective to treat the C. pneumoniae infection in macrophages or foam cells in the patient.

In any of the foregoing aspects, the dosage of the rifamycin analogue in the metal complex or formulation is normally about 0.001 to 100 mg/day. The metal complex or formulation may be given daily (e.g., a single oral dose of 2.5 to 25 mg/day) or less frequently (e.g., a single oral dose of 5, 12.5, or 25 mg/week). Treatment may be for one day to one year, or longer. In one embodiment, a metal complex or formulation is administered such that the rifamycin analogue is at an initial does of 2.5 to 100 mg for one to seven consecutive days, followed by a maintenance dose of 0.005 to 10 mg once every one to seven days for one month, one year, or even for the life of the patient.

If desired, a metal complex or formulation may be administered in conjunction with one or more additional agents such as anti-inflammatory agents (e.g., non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate) and steroids (e.g., cortisone, dexamethasone, hydrocortisone, methylprednisolone, prednisolone, prednisone, triamcinolone)), antibacterial agents (e.g., azithromycin, clarithromycin, erythromycin, gatifloxacin, levofloxacin, amoxicillin, or metronidazole), platelet aggregation inhibitors (e.g., abciximab, aspirin, cilostazol, clopidogrel, dipyridamole, eptifibatide, ticlopidine, or tirofiban), anticoagulants (e.g., dalteparin, danaparoid, enoxaparin, heparin, tinzaparin, or warfarin), antipyretics (e.g., acetaminophen), or lipid lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These additional agents may be administered within 14 days, 7 days, 1 day, 12 hours, or 1 hour of administration of the metal complex or formulation, or simultaneously therewith. The additional therapeutic agents may be present in the same or different pharmaceutical compositions as the metal complex or formulation. When present in different pharmaceutical compositions, different routes of administration may be used. For example, the metal complex or formulation may be administered orally, while a second agent may be administered by intravenous, intramuscular, or subcutaneous injection.

The invention also features a stent coated with a metal complex or formulation of the invention. The stent can be, e.g., a wire mesh tube used to hold open an artery. Stents are typically inserted following angioplasty.

The invention also features methods and compositions for treating or preventing an ear infection in a patient by topically administering to the affected otic area (e.g., the tympanic membrane or the external auditory canal of the ear) of the patient a pharmaceutical composition including a therapeutically effective amount of a metal complex or formulation of the invention. The compositions and methods of the invention can also be used to treat or prevent infections that result from surgery.

The invention also features a pharmaceutical composition suitable for topical administration to the ear of a patient containing a metal complex or formulation of the invention and a pharmaceutically-acceptable excipient, administered at a dose capable of reducing the infection in the patient. According to this invention, the rifamycin analogue can be in the amount between 0.001% and 5% weight/volume (w/v), preferably 0.01% and 3% w/v, more preferably 0.1% and 1% w/v, or most preferably 0.1% and 0.4% w/v. The metal complex or formulation may also be impregnated in a porous media (for example, an ear wick such as a sponge, gauze, cotton, or hydrocellulose), which is suitable for insertion into the ear of a patient. If desired, the composition may also include one or more penetration enhancers (e.g., alcohols, polyols, sulfoxides, esters, ketones, amides, oleates, surfactants, alkanoic acids, lactam compounds, alkanols, or admixtures thereof).

In another aspect, the invention also features a method for treating or preventing the development of an ear infection in a patient by administering a metal complex or formulation of the invention. The metal complex or formulation can be administered to the infected ear by means of drops or by the insertion of a rifamycin-impregnated porous media into the external ear canal to the tympanic membrane. Ear infections that can be treated using the methods and composition of the invention include otitis media and otitis extema. Types of otitis media amenable to treatment include, for example, acute otitis media, otitis media with effusion, and chronic otitis media. Types of otitis externa include acute otitis externa, chronic otitis externa, and malignant otitis externa. A rifamycin of the invention is administered to the ear (e.g., the tympanic membrane or the external auditory canal of the ear) to treat or prevent bacterial infections associated with otitis media (e.g., an infection of H. influenza, M. catarhalis, or S. pneumoniae) or in otitis externa (e.g., an infection of S. intermedius, Streptococcus spp. Pseudomonas spp., Proteus spp., or E. coli).

The methods and compositions of the invention are also useful to treat infections associated with otic surgical procedures such as tympanoplasty, stapedectomy, removal of tumors, or cochlear implant surgery. The compositions may also be used prophylactically, prior to therapies or conditions that can cause ear infections. Compositions containing a metal complex or formulation of the invention can therefore be applied to an area of the ear to which the surgical intervention will be performed, within at least seven days (before or after) of the surgical intervention. When treating a patient affected with otitis externa, an acidification therapy involving the administration of an acetic acid solution to the ear of the patient may also be performed.

Typically, patients are administered one to four drops of the composition of the invention having a rifamycin analogue in a total amount between 0.001% and 5% w/v, preferably 0.01% and 3% w/v, more preferably 0.1% and 1% w/v, or most preferably 0.1% and 0.4% w/v. The composition may be given daily (e.g., once, twice, three times, or four times daily) or less frequently (e.g., once every other day, or once or twice weekly). Treatment may be for 1 to 21 days, desirably 1 to 14 days, or even 3 to 7 days. Additional therapeutic agents, such as anti-inflammatory agents (e.g., non-steroidal anti-inflammatory or steroid), anesthetics, zinc salts, or other antimicrobial agents, can also be administered with the metal complex or formulation of the invention. Non-steroidal anti-inflammatory agents include, for example, detoprofen, diclofenac, diflunisal, etodolac, fenoprofen, flurbiprofen, indomethacin, ketoprofen, mechlofenameate, mefenamic acid, meloxicam, nabumeone, naproxen sodium, oxaprozin, piroxicam, sulindac, tolmeting, celecoxib, rofecoxib, choline salicylate, salsate, sodium salicylate, magnesium salicylate, aspirin, ibuprofen, paracetamol, acetaminophen, and pseudoephedrine and steroids include, for example, hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone. Anesthetics according to the invention can be, for example, benzocaine, butamben picrate, tetracaine, dibucaine, prilocaine, etidocaine, mepivacaine, bupivicaine, and lidocaine. A zinc salt can be zinc sulfate, zinc chloride, zinc acetate, zinc phenol sulfonate, zinc borate, zinc bromide, zinc nitrate, zinc glycerophosphate, zinc benzoate, zinc carbonate, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, and zinc glycinate, and antimicrobial agents according to the invention include, for example, amoxillin, erythromycin, azithromycin, clarithromycin, gentamicin, tobramycin, ciprofloxaxin, norfloxacin, gatifloxacin, ofloxacin, levofloxacin, moxifloxacin, metronidazole, lomefloxacin, ciprofloxacin, natamycin, neomycin, polymyxin B, gentamycin, trovafloxacin, grepafloxacin, sulfacetamide, tetracycline, gramicidin, chloremphenicol, bacitracin, and gramicidin. These additional therapeutic agents can be present in the same or different pharmaceutical compositions as the metal complex or formulation. When a therapeutic agent is present in a different pharmaceutical composition, different routes of administration may be used. The metal complex or formulation and and the second therapeutic agent, for example, may also be administered within 24 hours of each other, and an anti-inflammatory agent, for example, may be administered orally, or by intravenous, intramuscular, or subcutaneous injection.

To increase the efficacy of the topically administered composition, it is desirable that the amount of debris and granulation tissue are reduced in the infected ear of the patient at least one hour prior to the administration of the rifamycin and at least once a day. Debris can be removed, for example, by suction, irrigation with a solution containing hydrogen peroxide, cauterization, or by manual techniques employing microinstruments and microscope. Reduction in the amount of granulation tissue in the infected ear may be performed by means of cautering, or by the administration of a steroid.

The invention also features a pharmaceutical pack containing (i) a metal complex or formulation of the invention in an amount effective to treat a patient having an ear infection; and (ii) instructions for administering the metal complex or formulation to the ear of a patient. The invention also features a container containing a metal complex or formulation of the invention and a pharmaceutical excipient suitable for topical administration to the ear. If desired, an applicator for applying the composition to the ear may also be included. Desirably, a rifamycin analogue is present in the metal complex or formulation in the amount between 0.001% and 5% weight/volume (w/v), preferably 0.01% and 3% w/v, more preferably 0.1% and 1% w/v, or most preferably 0.1% and 0.4% w/v and is present in amounts sufficient to treat for at least 1, 3, 5, 7, 10, 14, or 21 days. A penetration enhancer may also be added (e.g., alcohols, polyols, sulfoxides, esters, ketones, amides, oleates, surfactants, alkanoic acids, lactam compounds, alkanols, or admixtures thereof).

The invention also features a method for treating chronic gastritis, gastric ulcer, or duodenal ulcer associated with an infection of H. pylori, or preventing the disease or infection, in a patient. The method includes the step of orally administering to the patient an effective amount of a metal complex or formulation of the invention to treat the patient. The metal complex or formulation is normally administered at about 1 to 1000 mg rifamycin analogue/day (desirably about 1 to 100 mg/day, more desirably about 5 to 50 mg/day, and even more desirably about 5 to 25 mg/day). The metal complex or formulation may be given daily (e.g., once, twice, three times, or four times daily) or less frequently (e.g., once every other day, or once or twice weekly). Treatment may be for 1 to 21 days, desirably 1 to 14 days or even 3 to 7 days. If desirable, a metal complex or formulation can be administered with a proton pump inhibitor (e.g., omeprazole, esomeprazole, lansoprazole, leminoprazole, pantoprazole or robeprazole), and/or bismuth preparation (e.g., colloidal bismuth subcitrate or bismuth subsalicylate).

The invention also features a pharmaceutical pack including (i) a metal complex or formulation of the invention in an amount effective to treat chronic gastritis, gastric ulcer, or duodenal ulcer associated with an infection of H. pylori in a patient; and (ii) instructions for administering to the patient. Desirably, the metal complex or formulation includes a rifamycin analogue in unit amounts of between 1 and 1000 mg (e.g, between 1 and 50 mg or between 5 and 5 mg), and is present in amounts sufficient to treat for at least 1, 3 5, 7, 10, 14, or 21 days. The pack may optionally include a proton pump inhibitor and/or bismuth preparation. In one embodiment, the metal complex or formulation is in a pharmaceutical composition with the proton pump inhibitor and/or bismuth preparation.

The invention also features a method for treating a patient having antibiotic-associated bacterial diarrhea or an infection of C. difficile, or preventing the disease or infection in the patient. The method includes the step of orally administering to the patient an effective amount of a metal complex or formulation of the invention to treat the patient. The metal complex or formulation includes a rifamycin analogue in unit amounts of about 1 to 1000 mg/day (desirably about 1 to 100 mg/day, more desirably about 5 to 50 mg/day, and even more desirably about 5 to 25 mg/day). The metal complex or formulation may be given daily (e.g., once, twice, three times, or four times daily) or less frequently (e.g., once every other day, or once or twice weekly). Treatment may be for 1 to 21 days, desirably 1 to 14 days or even 3 to 7 days. In one embodiment, a metal complex or formulation is administered at an initial dose of between 5 and 100 mg of rifamycin analogue, followed by subsequent doses of between 1 and 50 mg for 3 to 7 days. A single dose (e.g., in a dosage of between 5 and 50 mg) can also be employed in the method of the invention. If desirable, a metal complex or formulation can be administered with a second antibiotic (e.g., metronidazole).

The invention also features a pharmaceutical pack including (i) a metal complex or formulation of the invention in an amount effective to treat a patient having antibiotic-associated bacterial diarrhea or an infection of C. difficile; and (ii) instructions for administering to the patient for treating or preventing a C. difficile infection. Desirably, the metal complex or formulation includes a rifamycin analogue in unit amounts of between 1 and 1000 mg (e.g, between 1 and 50 mg or between 5 and 5 mg), and is present in amounts sufficient to treat for at least 1, 3 5, 7, 10, 14, or 21 days.

The invention features a method for treating a patient having an infection of C. trachomatis or N. gonorrhoeae. The method includes the step of administering to the patient a single oral dose of a metal complex or formulation of the invention in an amount effective to treat the patient.

The invention also features a pharmaceutical pack that includes (i) a single oral dose of a metal complex or formulation of the invention in an amount effective to treat a patient having an infection of C. trachomatis or N. gonorrhoeae; and (ii) instructions for administering the single oral dose to the patient. Desirably, the dose is in an amount between 0.1 and 100 mg of rifamycin analogue (e.g., between 1 and 50 mg or between 5 and 25 mg).

The invention also features a method of treating a patient having a chronic disease associated with a bacterial infection caused by bacteria capable of establishing a cryptic phase. This method includes the step of administering to a patient a metal complex or formulation of the invention for a time and in an amount sufficient to treat the cryptic phase of the bacterial infection. The chronic disease may be an inflammatory disease. Examples of inflammatory diseases include but are not limited to asthma, coronary artery disease, arthritis, conjunctivitis, lymphogranuloma venerum (LGV), cervicitis, and salpingitis. The chronic disease can also be an autoimmune disease (e.g., systemic lupus erythematosus, diabetes mellitus, graft versus host disease).

The invention further features a method of treating the non-replicating, cryptic phase of a bacterial infection. This method includes the step of administering to a patient a metal complex or formulation of the invention for a time and in an amount sufficient to treat the cryptic phase of the bacterial infection.

The invention also features a method of treating a bacterial infection in a patient by (a) treating the replicating phase or the elementary body phase of the chlamydial life cycle by administering an anti-bacterial agent to the patient for a time and an amount sufficient to treat the replicating phase or elementary body phase, and (b) treating the cryptic phase of the infection by administering to the patient a metal complex or formulation of the invention, wherein the administering is for a time and in an amount sufficient to treat the cryptic phase of the infection.

In a related aspect, the invention features a method of treating a chronic disease associated with a persistent intracellular bacterial infection or treating the persistent bacterial infection itself by administering a metal complex or formulation of the invention. As used herein, monotherapy is defined as a therapy in which the metal complex or formulation of the invention is the only antibacterial agent present in amounts sufficient to treat the cryptic phase of the infection.

In preferred embodiments of any of the foregoing aspects, the persistent intracellular bacterial infection is caused by one of the following: Chlamydia spp. (e.g., C. trachomatis, C. pneumoniae, C. psittaci, C. suis, C. pecorum, C. abortus, C. caviae, C. felis, C. muridarum), N. hartmannellae, W. chondrophila, S. negevensis, or P. acanthamoeba.

The time sufficient to treat the cryptic phase of the bacterial infection ranges from one week to one year, but it can also be extended over the lifetime of the individual patient, if necessary. In more preferable embodiments, the duration of treatment is at least 30 days, at least 45 days, at least 90 days, or at least 180 days. Ultimately, it is most desirable to extend the treatment for such a time that the intracellular bacterial infection is no longer detected.

In yet another aspect, the invention features a method of preventing, stabilizing, or inhibiting the growth of bacteria, or killing bacteria. The method involves contacting bacteria or a site susceptible to bacterial growth with a metal complex or formulation of the invention.

The complexes and formulations of the present invention can be used to treat, stabilize or prevent a bacterial infection in an animal. In this method, the step of contacting bacteria or a site susceptible to bacterial infection (e.g., a site in or on the body of animal) with the metal complex or formulation of the invention includes administering to the animal the metal complex or formulation in an amount sufficient to treat, stabilize, or prevent the bacterial infection in the animal.

The animal can be a human, an animal of veterinary interest (e.g., cow, horse, dog, pig, sheep, cat, or bird), or any other species.

The bacteria to be targeted by a metal complex or formulation of the invention can be, for example, one of the following: Acinetobacter calcoaceticus, A. haemolyticus, Aeromonas hydrophilia, Bacteroides fragilis, B. distasonis, Bacteroides 3452A homology group, B. vulgatus, B. ovalus, B. thetaiotaomicron, B. uniformis, B. eggerthii, B. splanchnicus, Branhamella catarrhalis, Campylobacter fetus, C. jejuni, C. coli, Citrobacter freundii, Clostridium difficile, C. diphtheriae, C. ulcerans, C. accolens, C. afermentans, C. amycolatum, C. argentorense, C. auris, C. bovis, C. confusum, C. coyleae, C. durum, C. falsenii, C. glucuronolyticum, C. imitans, C. jeikeium, C. kutscheri, C. kroppenstedtii, C. lipophilum, C. macginleyi, C. matruchoti, C. mucifaciens, C. pilosum, C. propinquum, C. renale, C. riegelii, C. sanguinis, C. singulare, C. striatum, C. sundsvallense, C. thomssenii, C. urealyticum, C. xerosis, Enterobacter cloacae, E. aerogenes, Enterococcus avium, E. casseliflavus, E. cecorum, E. dispar, E. durans, E. faecalis, E. faecium, E. flavescens, E. gallinarum, E. hirae, E. malodoratus, E. mundtii, E. pseudoavium, E. raffinosus, E. solitarius, Francisella tularensis, Gardnerella vaginalis, Helicobacter pylori, Kingella dentrificans, K. kingae, K. oralis, Klebsiella pneumoniae, K. oxytoca, Moraxella catarrhalis, M. atlantae, M. lacunata, M. nonliquefaciens, M. osloensis, M. phenylpyruvica, Morganella morganii, Parachlamydia acanthamoebae, Pasteurella multocida, P. haemolytica, Proteus mirabilis, Proteus vulgaris, Providencia alcalifaciens, P. rettgeri, P. stuartii, Serratia marcescens, Simkania negevensis, Streptococcus pneumoniae, S. agalactiae, S. pyogenes, Treponema pallidum, Vibrio cholerae, and V. parahaemolyticus. Accordingly, the invention features a method of treating infections by the bacteria specified above, among others.

In another embodiment, the bacterial infection to be treated or prevented by a metal complex or formulation of the invention can also be an intracellular infection by a facultative or obligate intracellular bacterium.

Facultative intracellular bacteria include, for example, Bordetella pertussis, B. parapertussis, B. bronchiseptica, Burkholderia cepacia, Escherichia coli, Haemophilus actinomycetemcomitans, H. aegyptius, H. aphrophilus, H. ducreyi, H. felis, H. haemoglobinophilus, H. haemolyticus, H. influenzae, H. paragallinarum, H. parahaemolyticus, H. parainfluenzae, H. paraphrohaemolyticus, H. paraphrophilus, H. parasuis, H. piscium, H. segnis, H. somnus, H. vaginalis, Legionella adelaidensis, L. anisa, L. beliardensis, L. birminghamensis, L. bozemanii, L. brunensis, L. cherrii, L. cincinnatiensis, Legionella drozanskii L. dumoffii, L. erythra, L. fairfieldensis, L. fallonii, L. feeleii, L. geestiana, L. gormanii, L. gratiana, L. gresilensis, L. hackeliae, L. israelensis, L. jordanis, L. lansingensis, Legionella londiniensis L. longbeachae, Legionella lytica L. maceachernii, L. micdadei, L. moravica, L. nautarum, L. oakridgensis, L. parisiensis, L. pittsburghensis, L. pneumophila, L. quateirensis, L. quinlivanii, L. rowbothamii, L. rubrilucens, L. sainthelensi, L. santicrucis, L. shakespearei, L. spiritensis, L. steigerwaltii, L. taurinensis, L. tucsonensis, L. wadsworthii, L. waltersii, L. worsleiensis, Listeria denitrificans, L. grayi, L. innocua, L. ivanovii, L. monocytogenes, L. seeligeri, L. welshimeri, Mycobacterium abscessus, M. africanum, M. agri, M. aichiense, M. alvei, M. asiaticum, M, aurum, M. austroafricanum, M. avium, M. bohemicum, M. bovis, M. branderi, M. brumae, M. celatum, M. chelonae, M. chitae, M. chlorophenolicum, M. chubuense, M. confluentis, M. conspicuum, M. cookii, M. diernhoferi, M. doricum, M. duvalii, M. elephantis, M. fallax, M. farcinogenes, M. flavescens, M. fortuitum, M. frederiksbergense, M. gadium, M. gastri, M. genavense, M. gilvum, M. goodii, M. gordonae, M. haemophilum, M. hassiacum, M. heckeshornense, M. heidelbergense, M. hiberniae, M. immunogenum, M. intracellular, M. interjectum, M. intermedium, M. kansasii, M. komossense, M. kubicae, M. lentiflavum, M. leprae, M. lepraemurium, M. luteum, M. madagascariense, M. mageritense, M. malmoense, M. marinum, M. microti, M. moriokaense, M. mucogenicum, M. murale, M. neoaurum, M. nonchromogenicum, M. novocastrense, M. obuense, M. parafortuitum, M. paratuberculosis, M. peregrinum, M. phage, M. phlei, M. porcinum, M. poriferae, M. pulveris, M. rhodesiae, M. scrofulaceum, M. senegalense, M. septicum, M. shimoidei, M. simiae, M. smegmatis, M. sphagni, M. szulgai, M. terrae, M. thermoresistibile, M. tokaiense, M. triplex, M. triviale, M. tuberculosis, M. tusciae, M. ulcerans, M. vaccae, M. wolinskyi, M. xenopi, Neisseria animalis, N. canis, N. cinerea, N. denitrificans, N. dentiae, N. elongata, N. flava, N. flavescens, N. gonorrhoeae, N. iguanae, N. lactamica, N. macacae, N. meningitidis ,N. mucosa, N. ovis, N. perflava, N. pharyngis var. flava, N. polysaccharea, N. sicca, N. subflava, N. weaveri, Pseudomonas aeruginosa, P. alcaligenes, P. chlororaphis, P. luorescens, P. luteola, P. mendocina, P. monteilii, P. oryzihabitans, P. pertocinogena, P. pseudalcaligenes, P. putida, P. stutzeri, Salmonella bacteriophage, S. bongori, S. choleraesuis, S. enterica, S. enteritidis, S. paratyphi, S. typhi, S. typhimurium, S. typhimurium, S. typhimurium, S. typhimurium bacteriophage, Shigella boydii, S. dysenteriae, S. flexneri, S. sonnei, Staphylococcus arlettae, S. aureus, S. auricularis, S. bacteriophage, S. capitis, S. caprae, S. carnosus, S. caseolyticus, S. chromogenes, S. cohnii, S. delphini, S. epidermidis, S. equorum, S. felis, S. fleurettii, S. gallinarum, S. haemolyticus, S. hominis, S. hyicus, S. intermedius, S. kloosii, S. lentus, S. lugdunensis, S. lutrae, S. muscae, S. mutans, S. pasteuri, S. phage, S. piscifermentans, S. pulvereri, S. saccharolyticus, S. saprophyticus, S. schleiferi, S. sciuri, S. simulans, S. succinus, S. vitulinus, S. warneri, S. xylosus, Ureaplasma urealyticum, Yersinia aldovae, Y. bercovieri, Y. enterocolitica, Y. frederiksenii, Y. intermedia, Y. kristensenii, Y. mollaretii, Y. pestis, Y. philomiragia, Y. pseudotuberculosis, Y. rohdei, and Y. ruckeri. Accordingly, the invention features a method of treating infections by the facultative intracellular bacteria specified above, among others.

Obligate intracellular bacteria include, for example, Anaplasma bovis, A. caudatum, A. centrale, A. marginale A. ovis, A. phagocytophila, A. platys, Bartonella bacilliformis, B. clarridgeiae, B. elizabethae, B. henselae, B. henselae phage, B. quintana, B. taylorii, B. vinsonii, Borrelia afzelii, B. andersonii, B. anserina, B. bissettii, B. burgdorferi, B. crocidurae, B. garinii, B. hermsii, B. japonica, B. miyamotoi, B. parkeri, B. recurrentis, B. turdi, B. turicatae, B. valaisiana, Brucella abortus, B. melitensis, Chlamydia pneumoniae, C. psittaci, C. trachomatis, Cowdria ruminantium, Coxiella burnetii, Ehrlichia canis, E. chaffeensis, E. equi, E. ewingii, E. muris, E. phagocytophila, E. platys, E. risticii, E. ruminantium, E. sennetsu, Haemobartonella canis, H. felis, H. muris, Mycoplasma arthriditis, M. buccale, M. faucium, M. fermentans, M. genitalium, M. hominis, M. laidlawii, M. lipophilum, M. orale, M. penetrans, M. pirum, M. pneumoniae, M. salivarium, M. spermatophilum, Rickettsia australis, R. conorii, R. felis, R. helvetica, R. japonica, R. massiliae, R. montanensis, R. peacockii, R. prowazekii, R. rhipicephali, R. rickettsii, R. sibirica, and R. typhi. Accordingly, the invention features a method of treating infections by the obligate intracellular bacteria specified above, among others.

In another aspect, the invention features a pharmaceutical composition that includes a metal complex or formulation described herein in any pharmaceutically acceptable form, including isomers such as diastereomers and enantiomers, salts, solvates, and polymorphs thereof. In various embodiments, the composition includes a compound of the invention along with a pharmaceutically acceptable carrier or diluent.

In any of the above aspects, desirable rifamycin analogues include compounds 7-10, shown below.

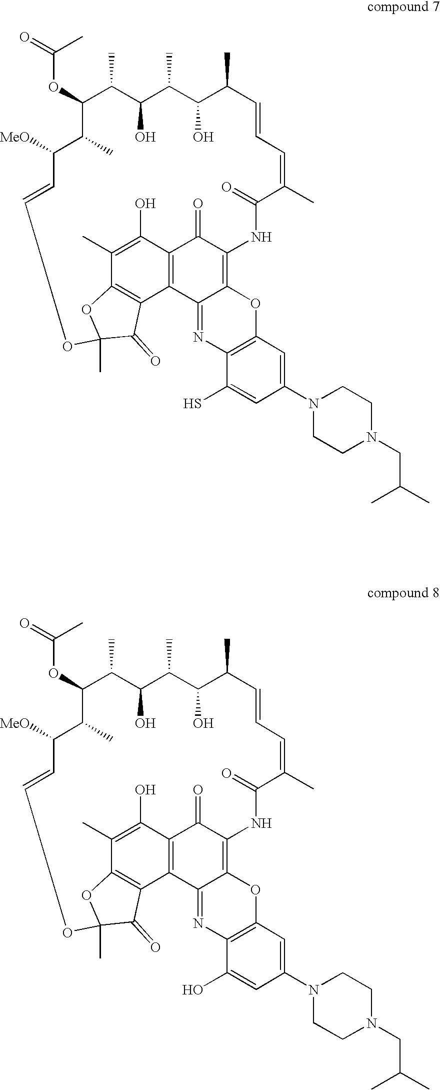

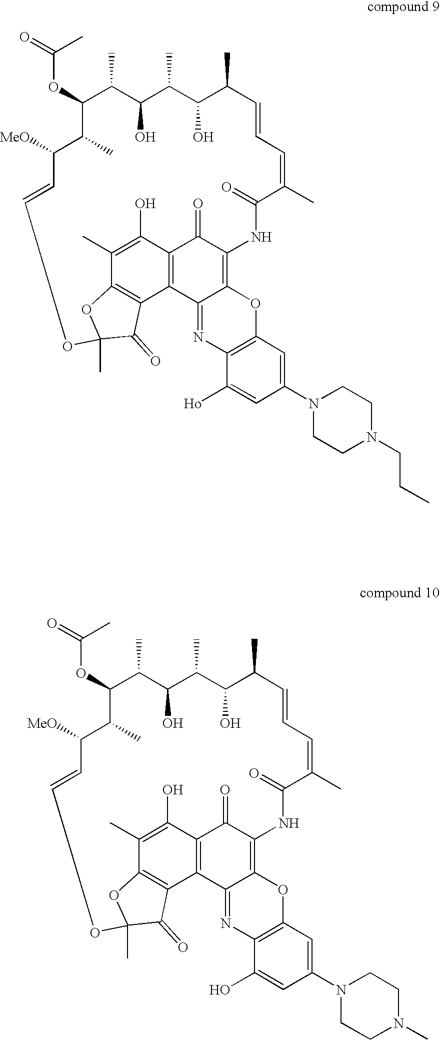

By “alkyl” is meant a branched or unbranched saturated hydrocarbon group, desirably having from 1 to 10 carbon atoms. An alkyl may optionally include monocyclic, bicyclic, or tricyclic rings, in which each ring desirably has three to six members. The alkyl group may be substituted or unsubstituted. Exemplary substituents include alkoxy, aryloxy, sulfhydryl, alkylthio, arylthio, halogen, hydroxy, fluoroalkyl, perfluoralkyl, amino, aminoalkyl, disubstituted amino, quaternary amino, hydroxyalkyl, carboxyalkyl, and carboxyl groups.

In various embodiments of the invention the alkyl group is of 1 to 10 carbon atoms. Exemplary substituents include methyl; ethyl; n-propyl; isopropyl; n-butyl; iso-butyl; sec-butyl; tert-butyl; pentyl; cyclopropyl; cyclobutyl; cyclopentyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; hexyl; heptyl; cyclohexyl; cycloheptyl; and cyclooctyl.

By “aryl” is meant an aromatic group having a ring system comprised of carbon atoms with conjugated π electrons (e.g., phenyl). The ring of the aryl group is desirably 6 to 18 atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halogen, fluoroalkyl, carboxyl, carboxyalkyl, amino, aminoalkyl, monosubstituted amino, disubstituted amino, and quaternary amino groups.

By “fluoroalkyl” is meant an alkyl group that is substituted with a fluorine.

By “perfluoroalkyl” is meant an alkyl group consisting of only carbon and fluorine atoms.

By “carboxyalkyl” is meant a chemical moiety with the formula —(R)—COOH, wherein R is an alkyl group.

By “hydroxyalkyl” is meant a chemical moiety with the formula —(R)—OH, wherein R is an alkyl group.

By “alkoxy” is meant a chemical substituent of the formula —OR, wherein R is an alkyl group.

By “aryloxy” is meant a chemical substituent of the formula —OR, wherein R is an aryl group.

By “alkylthio” is meant a chemical substituent of the formula —SR, wherein R is an alkyl group.

By “arylthio” is meant a chemical substituent of the formula —SR, wherein R is an aryl group.

By “quaternary amino” is meant a chemical substituent of the formula —(R)—N(R′)(R″)(R′″)⁺, wherein R, R′, R″, and R′″ are each independently an alkyl, alkene, alkyne, or aryl group. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

By “bacterial infection” is meant the invasion of a host animal by pathogenic bacteria. For example, the infection may include the excessive growth of bacteria that are normally present in or on the body of an animal or growth of bacteria that are not normally present in or on the animal. More generally, a bacterial infection can be any situation in which the presence of a bacterial population(s) is damaging to a host animal. Thus, an animal is “suffering” from a bacterial infection when an excessive amount of a bacterial population is present in or on the animal's body, or when the presence of a bacterial population(s) is damaging the cells or other tissue of the animal. The bacterial infection may be due to gram positive and/or gram negative bacteria.

By “intracellular infection” is meant an infection by any facultative or obligate intracellular bacteria.

By “obligate intracellular bacteria” is meant bacteria which must use an intracellular location (e.g., a host cell) in order to replicate.

By “facultative intracellular bacteria” is meant bacteria which are able to survive within an intracellular location (e.g., a host cell), but do not require an intracellular environment to replicate.

By “administration” or “administering” is meant a method of giving one or more unit doses of an antibacterial pharmaceutical composition to an animal (e.g., topical, oral, intravenous, intraperitoneal, or intramuscular administration). The method of administration may vary depending on various factors, e.g., the components of the pharmaceutical composition, site of the potential or actual bacterial infection, bacteria involved, and severity of the actual bacterial infection.

By “metal-rifamycin analogue complex” is meant a molecule containing bonds between a metal selected from the group consisting of Groups I (A, B), II (A, B), III (A, B), IV (A, B), V (A, B), VIA, VIIA, and VIII, and a rifamycin analogue of formula I. Metal-rifamycin analogue complexes include chelates formed between a compound of formula I and a metal and salts thereof. Metal-rifamycin analogue complexes may be described by formula III, or may be a salt thereof. The atoms within the rifamycin analog available for bond formation with a metal center are any with a lone pair (e.g., O, S, or N) available for forming a dative bond to the metal center. In some instances one or more protons are removed from the rifamycin analogue in the process of forming a metal complex. In such instances, the rifamycin analogue of formula III represents a fragment described by formulas I or II minus any removed protons. For example, a hydroxyl or sulfhydryl group may be deprotonated prior to or after forming a bond with a metal center. Thus, an alcohol-metal or thiol-metal bond becomes an alkoxide-metal or thiolate-metal bond upon removal of a proton. Complexed rifamycin analogues exhibit improved solubility in water. Between pH 5 and 9, complexed rifamycin analogues have a solubility of at least 2.0 μg/mL.

By “rifamycin analogue” is meant a compound of formula I.

By “metal salt” is meant any compound that results from replacement of part or all of the acid hydrogen of an acid by a metal (e.g., sodium chloride).

By “minimum inhibitory concentration” is meant the minimum concentration of a compound or formulation of the present invention required to inhibit greater than 99% of the bacterial growth in the in vitro assay described herein.

By “atherosclerosis” is meant the progressive accumulation of smooth muscle cells, immune cells (e.g., lymphocytes, macrophages, or monocytes), lipid products (e.g., lipoproteins, or cholesterol), cellular waste products, calcium, or other substances within the inner lining of an artery, resulting in the narrowing or obstruction of the blood vessel and the development of atherosclerosis-associated diseases. Atherosclerosis is typically manifested within large and medium-sized arteries, and is often characterized by a state of chronic inflammation within the arteries.

By “atherosclerosis-associated disease” is meant any disorder that is caused by or is associated with atherosclerosis. Typically, atherosclerosis of the coronary arteries commonly causes coronary artery disease, myocardial infarction, coronary thrombosis, and angina pectoris. Atherosclerosis of the arteries supplying the central nervous system frequently provokes strokes and transient cerebral ischemia. In the peripheral circulation, atherosclerosis causes intermittent claudication and gangrene and can jeopardize limb viability. Atherosclerosis of an artery of the splanchnic circulation can cause mesenteric ischemia. Atherosclerosis can also affect the kidneys directly (e.g., renal artery stenosis).

A patient who is being treated for an atherosclerosis-associated disease is one who a medical practitioner has diagnosed as having such a disease. Diagnosis may be by any suitable means. Methods for diagnosing atherosclerosis by measuring systemic inflammatory markers are described, for example, in U.S. Pat. No. 6,040,147, hereby incorporated by reference. Diagnosis and monitoring may employ an electrocardiogram, chest X-ray, echocardiogram, cardiac catheterization, ultrasound (for the measurement of vessel wall thickness), or measurement of blood levels of CPK, CPK-MB, myoglobin, troponin, homocysteine, or C-reactive protein. A patient in whom the development of an atherosclerosis-associated disease is being prevented is one who has not received such a diagnosis. One in the art will understand that these patients may have been subjected to the same tests (electrocardiogram, chest X-ray, etc.) or may have been identified, without examination, as one at high risk due to the presence of one or more risk factors (e.g., family history, hypertension, diabetes mellitus, high cholesterol levels). Thus, prophylactic administration of a metal complex or formulation of the invention is considered to be preventing the development of an atherosclerosis-associated disease.

An atherosclerosis-associated disease has been treated or prevented when one or more tests of the disease (e.g., any of the those described above) indicate that the patient's condition has improved or the patient's risk reduced. In one example, a reduction in C-reactive protein to normal levels indicates that an atherosclerosis-associated disease has been treated or prevented.

An alternative means by which treatment or prevention is assessed includes determination of the presence of an infection of C. pneumoniae. Any suitable method may be employed (e.g., determination of C. pneumoniae in blood monocytes or in the atheroma itself (e.g., in macrophages or foam cells present in the fatty streak), or detection of C. pneumoniae DNA, RNA, or antibodies to C. pneumoniae in a biological sample from the patient).

By “debris” is meant the mucoid exudate or desquamated epithelium in an infected ear of a patient having an ear infection.

By “ear wick” is meant a sponge, cotton, gauze, compressed hydroxycellulose, or any other material used to increase the penetration of rifamycin to the infected otic area. The ear wick is typically inserted into the canal under direct vision. Its presence helps wick eardrops along the canal, hold the solution in contact with the skin of the canal, and apply pressure to the canal skin.

By “granulation tissue” is meant the highly vascularized tissue that replaces the initial fibrin clot in a wound. Vascularization is a result of an ingrowth of capillary endothelium from the surrounding vasculature. The tissue is also rich in fibroblasts and leucocytes.

“Antibiotic-associated bacterial diarrhea” means the condition wherein antibiotic therapy disturbs the balance of the microbial flora of the gut, allowing pathogenic organisms such as C. difficile to flourish. These organisms cause diarrhea. Antibiotic-associated bacterial diarrhea includes such conditions as C. difficile associated diarrhea (CDAD) and pseudomembranous colitis.

“Pseudomembranous colitis,” also known as pseudomembranous enterocolitis or enteritis, means the inflammation of the mucous membrane of both small and large intestine with the formation and passage of pseudomembranous material (composed of fibrin, mucous, necrotic epithelial cells and leukocytes) in the stools.

By “autoimmune disease” is meant a disease arising from an immune reaction against self-antigens and directed against the individual's own tissues. Examples of autoimmune diseases include but are not limited to systemic lupus erythematosus, rheumatoid arthritis, myasthenia gravis, and Graves' disease.

By “bacteria” is meant a unicellular prokaryotic microorganism that usually multiplies by cell division.

By “bacteria capable of establishing a cryptic phase” is meant any species whose life cycle includes a persistent, non-replicating, metabolically inactive phase. These species include but are not limited to C. trachomatis, C. neumoniae, C. psittaci, C. suis, C. pecorum, C. abortus, C. caviae, C. felis, C. muridarum, N. hartmannellae, W. chondrophila, S. negevensis, and P. acanthamoeba, as well as any other species described in Everett et al. (Int. J. Syst. Evol. Microbiol. 49:415-440, 1999), incorporated herein by reference.

By “chronic disease” is meant a disease that is inveterate, of long continuance, or progresses slowly, in contrast to an acute disease, which rapidly terminates. A chronic disease may begin with a rapid onset or in a slow, insidious manner but it tends to persist for several weeks, months or years, and has a vague and indefinite termination.

By “cryptic phase” is meant the latent or dormant intracellular phase of infection characterized by little or no metabolic activity. The non-replicating cryptic phase is often characteristic of persistent forms of intracellular bacterial infections.

By “elementary body phase” is meant the infectious phase of the bacterial life cycle characterized by the presence of elementary bodies (EBs). EBs are small (300-400 nm), infectious, spore-like forms which are metabolically inactive, non-replicating, and found most often in the acellular milieu. EBs possess a rigid outer membrane which protects them from a variety of physical insults such as enzymatic degradation, sonication and osmotic pressure.

By “immunocompromised” is meant a person who exhibits an attenuated or reduced ability to mount a normal cellular or humoral defense to challenge by infectious agents, e.g., viruses, bacterial, fungi, and protozoa. Persons considered immunocompromised include malnourished patients, patients undergoing surgery and bone narrow transplants, patients undergoing chemotherapy or radiotherapy, neutropenic patients, HIV-infected patients, trauma patients, bum patients, patients with chronic or resistant infections such as those resulting from myelodysplastic syndrome, and the elderly, all of who may have weakened immune systems.

By “inflammatory disease” is meant a disease state characterized by (1) alterations in vascular caliber that lead to an increase in blood flow, (2) structural changes in the microvasculature that permit the plasma proteins and leukocytes to leave the circulation, and (3) emigration of the leukocytes from the microcirculation and their accumulation in the focus of injury. The classic signs of acute inflammation are erythema, edema, tenderness (hyperalgesia), and pain. Chronic inflammatory diseases are characterized by infiltration with mononuclear cells (e.g., macrophages, lymphocytes, and plasma cells), tissue destruction, and fibrosis. Non-limiting examples of inflammatory disease include asthma, coronary artery disease, arthritis, conjunctivitis, lymphogranuloma venerum, and salpingitis.

By “intracytoplasmic inclusion” is meant a replicating reticulate body (RB) that has no cell wall. Such inclusions may be detected, for example, through chlamydiae sample isolation and propagation on a mammalian cell lines, followed by fixing and staining using one of a variety of staining methods including Giemsa staining, iodine staining, and immunofluorescence. These inclusions have a typical round or oval appearance.

By “persistent bacterial infection” is meant an infection that is not completely eradicated through standard treatment regimens using anti-bacterial agents. Persistent bacterial infections are caused by bacteria capable of establishing a cryptic or latent phase of infection and may be classified as such by culturing the bacteria from a patient and demonstrating bacterial survival in vitro in the presence of anti-bacterial agents or by determination of anti-bacterial treatment failure in a patient. As used herein, a persistent infection in a patient includes any recurrence of chlamydial infection, after receiving anti-bacterial treatment, from the same species (e.g., C. trachomatis) more than two times over the period of two or more years or the detection of the cryptic phase of the infection in the patient by the methods described. An in vivo persistent infection can be identified through the use of a reverse transcriptase polymerase chain reaction (RT-PCR) to demonstrate the presence of 16S rRNA transcripts in bacterially infected cells after treatment with anti-bacterial agents (Antimicrob. Agents Chemother. 12:3288-3297, 2000).

By “replicating phase” is meant the phase of the bacterial cell cycle characterized by the presence of an RB. The RB is the actively replicating form of the Chlamydia. It contains no cell wall and is detected as an inclusion in the cell.

By “bolus” injection or administration is meant an intravenous administration of the metal-rifamycin analogue complex wherein a dose of greater than 2 mg of the metal-rifamycin analogue complex is administered over a period of less than one hour.

By “infusion” is meant a continuous intravenous administration of the metal-rifamycin analogue complex over a period of greater than one hour wherein the metal-rifamycin analogue complex is administered at a constant rate.

By “aqueous solution” is meant a liquid that is greater than 40% water by volume and without undissolved solids above 0.5 microns in size. Desirably, in aqueous solutions of a metal-rifamycin analogue complex, the complex is completely dissolved.

Other features and advantages of the invention will be apparent from the following detailed description and from the claims.

### BRIEF DESCRIPTION OF THE DRAWINGS

FIG. 1 is a synthetic scheme depicting the synthesis of compound 7.

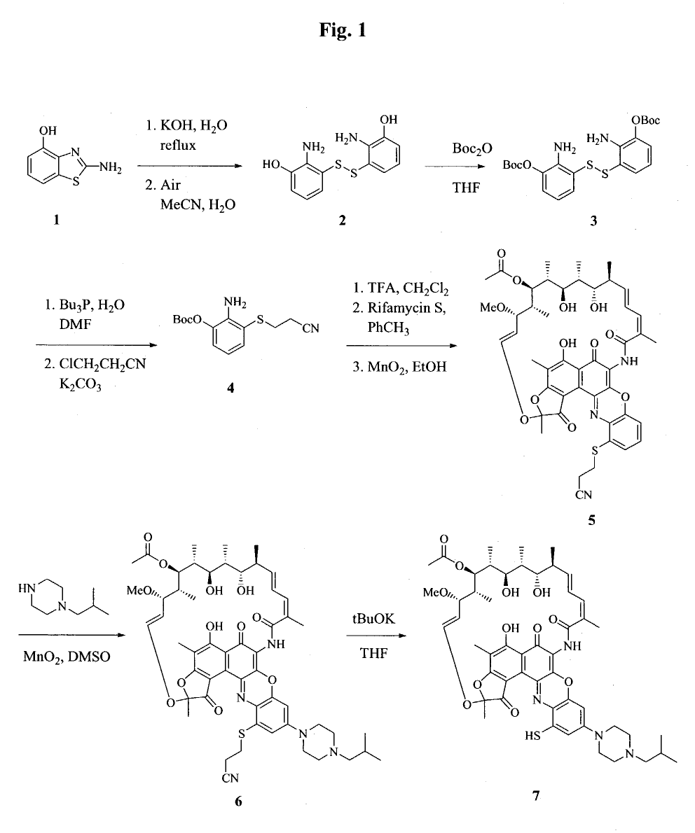

### DETAILED DESCRIPTION

We have discovered that rifamycin analogues formulated with metals and rifamycin analogues complexed with metals are useful for treating or preventing a variety of bacterial infections. The metal complexes and formulations of the present invention have two characteristic components: a metal formulated with, or complexed to, a rifamycin analogue of formula I:

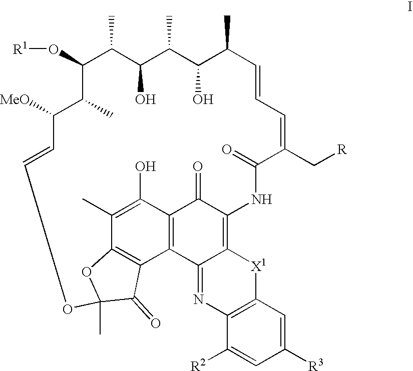

In formula I, X¹ represents an oxygen atom or a sulfur atom; R represents a hydrogen atom or hydroxyl group; R¹ represents acetyl or H, OH; R² represents a hydroxyl group or a sulfhydryl group; and R³ represents:

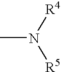

wherein each of R⁴ and R⁵ is, independently, an alkyl group having 1 to 7 carbon atoms, or R⁴ and R⁵ combine to form a 3-8 membered cyclic system,

or R³ represents a group expressed by the formula:

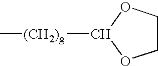

in which g represents an integer between 1 and 3,

or R³ represents a group expressed by the formula:

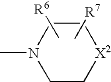

wherein each of R⁶ and R⁷ is, independently, a hydrogen atom or an alkyl group having 1 to 3 carbon atoms, X² represents an oxygen atom, a sulfur atom, or a carbonyl group,

or X² represents:

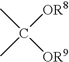

in which each of R⁸ and R⁹ is, independently, a hydrogen atom, or an alkyl group having 1 to 3 carbon atoms, or R⁸ and R⁹, in combination with each other, represent —(CH₂)k— in which k represents an integer between 1 and 4,

or X² represents:

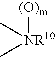

in which m represents 0 or 1, R¹⁰ represents a hydrogen atom, an alkyl group having 1 to 6 carbon atoms, or —(CH₂)ₙX³ in which n represents an integer between 1 and 4, and X³ represents an alkoxy group having 1 to 3 carbon atoms, a vinyl group, an ethynyl group,

or R¹⁰ represents:

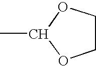

Formulations of the invention contain a metal salt added in an amount sufficient to reduce the minimum inhibitory concentration of the rifamycin analogue.

The minimum inhibitory concentration of the rifamycin analogue formulated with a metal salt is less than 95%, 90%, 80%, 70%, 60%, 50%, 40%, 30%, 20%, 10%, 5%, 1%, or 0.1% of the minimum inhibitory concentration of the rifamycin analogue formulated without a metal salt.

We have also identified metal complexes of rifamycin analogues which are useful for treating or preventing a variety of bacterial infections. These complexes are formed by the combination of a metal salt and a rifamycin analogue of formulas I or II.

We have identified a method of preventing, stabilizing, or inhibiting the growth of bacteria, or killing bacteria. The method involves contacting bacteria or a site susceptible to bacterial growth with a metal complex or formulation of the invention. Complexes and formulations of the present invention can be used to treat, stabilize, or prevent a bacterial infection in an animal. In this method, the step of contacting bacteria or a site susceptible to bacterial infection (e.g., a site in or on the body of an animal) with the metal complex or formulation of the invention includes administering to the animal the metal complex or formulation in an amount sufficient to treat, stabilize, or prevent the bacterial infection in the animal.

Rifamycin Analogues

The rifamycin analogues of formula I can be synthesized, for example, by the methods disclosed in U.S. Pat. Nos. 4,690,919, 4,983,602, 5,786,349, 5,981,522, and 4,859,661 and Chem. Pharm. Bull., 41:148, 1993, each of which is hereby incorporated by reference.

The synthesis of rifamycin derivatives of formula I in which R² is sulfhydryl or in which X¹ is a sulfur atom is provided in Example 3. The synthesis of rifamycin derivatives of formula I in which R² is sulfhydryl and in which X¹ is an oxygen atom is provided in Example 5.

Metal Salts

Metal salts of the present invention are used in the preparation of formulations that include a rifamycin analogue of formula I and a metal salt. Metal salts also are used as starting materials in the preparation of metal complexes of rifamycin analogues.

Metals of the present invention are selected for their ability to enhance the antibacterial properties of rifamycin analogues of formula I without introducing an unacceptable risk of metal-induced toxicity. Exemplary metals include, but are not limited to, aluminum, bismuth, cesium, calcium, chromium, cobalt, copper, gallium, gadolinium, germanium, gold, iridium, iron, magnesium, manganese, neodymium, nickel, palladium, platinum, potassium, rhenium, rhodium, rubidium, ruthenium, samarium, scandium, selenium, silver, sodium, tin, titanium, vanadium, and zinc.

For metal salts used in the invention, a salt is selected either for its commercial availability, synthetic utility in the preparation of metal complexes, or its biocompatibility in the formulation of metal salt-rifamycin analogue formulations. Examples of salts include but are not limited to acetate, adipate, alginate, ascorbate, aspartate, benzoate, benzenesulfonate, bisulfate, borate, butyrate, camphorate, camphorsulfonate, carbonate, citrate, cyclopentanepropionate, digluconate, dodecylsulfate, ethanesulfonate, fumarate, glucoheptanoate, glutamate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, oxide, palmitate, pamoate, pectinate, persulfate, 3-phenylpropionate, phosphate, picrate, pivalate, propionate, salicylate, suberate, succinate, sulfate, tartrate, thiocyanate, tosylate, undecanoate sulfate, bromide, chloride, fluoride, and iodide.

Metal-Rifamycin Analogue Complexes

Metal complexes of rifamycin analogues are prepared using standard methods known in the art. A commonly used method of preparing metal complexes is to combine a solution of metal salt with a solution of desired ligand. In the present invention, the desired ligand is a compound of formulas I or II. This type of reaction involves ligand metathesis, as shown in reaction 1. In reaction 1

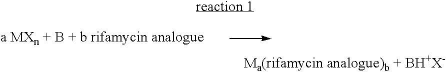

a and b describe the stoichiometry of the reaction, which is dictated by the reaction product, a metal complex of formula III. For reactions and complexes that require the deprotonation of a rifamycin analogue, a base, B (e.g., hydroxide, oxide, ammonia, or alkyl amines), may be added to promote the reaction. The formation of a transition metal complex is often accompanied by a color change. The formation of a metal complex is readily identifiable by any one of a variety of known techniques (e.g., Uv-vis, IR, NMR, mass spectroscopy, HPLC, cyclic voltammetry, or elemental analysis). The oxidation state of the metal in the complex of formula III can be controlled by the selection of starting materials containing the metal in the desired oxidation state. In some cases, the oxidation state of the metal in the complex of formula III is adjusted during the reaction by addition of a oxidizing agent (e.g., oxygen, ferrocenium salts, alky and aryl N-oxides, alkyl and aryl N-halides, permanganate, chromate, chlorine, bromine, iodine, hydrazines, or peroxides), or by addition of a reducing agent (e.g., hydrogen, metal-hydrides, lithium, sodium, potassium, calcium, zinc, or iron). In some cases, the desired oxidation state of the metal in the complex of formula III is obtained by using a mixture of metal salt starting materials. Further details are provided in Example 2.

While the rifamycin analogues are virtually insoluble in water at physiological pH, their metal complexes exhibit good solubility in water (see Example 6). As a result of their improved solubility, it is desirable to use the complexes to simplify the formulation or enhance the absorption of the rifamycin analogue. For example, the complexes can be used to prepare aqueous solutions for intravenous delivery or to enhance absorption when delivered orally. Metal-rifamycin analogue complexes can be used in accordance with any of the methods of administration described herein.

Assays

Compounds or formulations of the present invention can be screened for antibacterial activity by measuring their minimum inhibitory concentration (MIC), using standard MIC in vitro assays (see, for example, Tomioka et al., Antimicrob. Agents Chemother. 37:67, 1993). For example, agents can be screened against C. pneumoniae, C. trachomatis, M. tuberculosis (including multiple drug resistant strains), M. avium complex, or other intracellular infectious bacteria. Details of a standard MIC assay are provided in Example 4.

Therapy

Metal complexes or formulations of the invention may be administered by any appropriate route for treatment, stabilization, or prevention of a bacterial infection. These compounds may be administered to humans, domestic pets, livestock, or other animals with a pharmaceutically acceptable diluent, carrier, or excipient, in unit dosage form. Administration may be oral, topical, parenteral, intravenous, intra-arterial, subcutaneous, intramuscular, intracranial, intraorbital, ophthalmic, intraventricular, intracapsular, intraspinal, intracistemal, intraperitoneal, intranasal, aerosol, by suppositories, or by any other suitable route of administration.

We have identified a method of preventing, stabilizing, or inhibiting the growth of microbes, or killing microbes. The method involves contacting microbes or a site susceptible to microbial growth with a metal complex or formulation of the invention. Compounds of the present invention can be used to treat, stabilize or prevent a microbial infection in an animal. In this method, the step of contacting microbes or a site susceptible to microbial infection (e.g., a site in or on the body of an animal) with metal complex or formulation of the invention in an amount sufficient to treat, stabilize, or prevent the microbial infection in the animal.

In particular embodiments, a metal complex or formulation of the invention can be used to treat atherosclerosis or diseases associated therewith, sexually transmitted diseases caused, for example, by C. trachomatis or N. gonorrhoeae, otitis media and other ear infections, antibiotic-associated colitis, gastritis and ulcers associated with an infection of H. pylori, community-acquired pneumonia, upper and lower respiratory tract infections, skin and soft tissue infections, bone and joint infections, hospital-acquired lung infections, urinary tract infections, pyelonephritis, intra-abdominal infections, bacterial sepsis, would infections, peritonitis, osteomyelitis, infections after burns, pelvic inflammatory disease, and diseases associated with chronic infections.

Atherosclerosis and Other Diseases Associated with Chlamydial Infection

An association was previously reported in International Publication No. WO 98/50074 between the cryptic phase of a persistent chlamydial infection of body fluids and/or tissues and several chronic disease syndromes of previously unknown etiology in humans. To date, these diseases include, but are not limited to, atherosclerosis, multiple sclerosis, rheumatoid arthritis, inflammatory bowel disease, interstitial cystitis, fibromyalgia, autonomic nervous dysfunction (neural-mediated hypotension); pyoderma gangrenosum, and chronic fatigue syndrome.

As described in International Publication No. WO 98/50074, several lines of evidence have led to the establishment of a link between Chlamydia and a broad set of inflammatory, autoimmune, and immune deficiency diseases. These include (i) the association between the cryptic phase of a persistent chlamydial infection of body fluids and/or tissues and several chronic disease syndromes as described above, (ii) published evidence of an association between atherosclerosis and Chlamydia (Circulation 96:404-407, 1997), and (iii) an understanding of the impact the persistent infection established by the cryptic phase of chlamydial infections can have on infected cells and the immune system. Thus, the present invention describes methods for treating chronic diseases associated with the cryptic phase of a persistent chlamydial infection, such as autoimmune diseases, inflammatory diseases and diseases that occur in immuno-compromised individuals by treating the cryptic phase of the infection in an individual in need thereof, using a metal complex or formulation described herein. Progress of the treatment can be evaluated, using the diagnostic tests described herein, to determine the presence or absence of Chlamydia. Physical improvement in the conditions and symptoms typically associated with the disease to be treated can also be evaluated. Based upon these evaluating factors, the physician can maintain or modify the anti-bacterial therapy accordingly.

The therapies described herein can be used for the treatment of chronic immune and autoimmune diseases when patients are demonstrated to have a chlamydial load by the methods of detection described above. These diseases include, but are not limited to, chronic hepatitis, systemic lupus erythematosus, arthritis, thyroidosis, scleroderna, diabetes mellitus, Graves' disease, Beschet's disease, and graft versus host disease (graft rejection). The therapies of this invention can also be used to treat any disorders in which a chlamydial species is a factor or co-factor.

Thus, the present invention can be used to treat a range of disorders in addition to the above immune and autoimmune diseases when demonstrated to be associated with chlamydial infection by the methods of detection described herein; for example, various infections, many of which produce inflammation as primary or secondary symptoms, including, but not limited to, sepsis syndrome, cachexia, circulatory collapse and shock resulting from acute or chronic bacterial infection, acute and chronic parasitic and/or infectious diseases from bacterial, viral or fingal sources, such as a HIV, AIDS (including symptoms of cachexia, autoimmune disorders, AIDS dementia complex and infections) can be treated, as well as Wegners Granulomatosis.

Among the various inflammatory diseases, there are certain features that are generally agreed to be characteristic of the inflammatory process. These include fenestration of the microvasculature, leakage of the elements of blood into the interstitial spaces, and migration of leukocytes into the inflamed tissue. On a macroscopic level, this is usually accompanied by the familiar clinical signs of erythema, edema, tenderness (hyperalgesia), and pain. Inflammatory diseases, such as chronic inflammatory pathologies and vascular inflammatory pathologies, including chronic inflammatory pathologies such as aneurysms, hemorrhoids, sarcoidosis, chronic inflammatory bowel disease, ulcerative colitis, and Crohn's disease and vascular inflammatory pathologies, such as, but not limited to, disseminated intravascular coagulation, atherosclerosis, and Kawasaki's pathology are also suitable for treatment by methods described herein. The invention can also be used to treat inflammatory diseases such as coronary artery disease, hypertension, stroke, asthma, chronic hepatitis, multiple sclerosis, peripheral neuropathy, chronic or recurrent sore throat, laryngitis, tracheobronchitis, chronic vascular headaches (including migraines, cluster headaches and tension headaches) and pneumonia.

Treatable disorders when associated with Chlamydia infection also include, but are not limited to, neurodegenerative diseases, including, but not limited to, demyelinating diseases, such as multiple sclerosis and acute transverse myelitis; extrapyramidal and cerebellar disorders, such as lesions of the cortico spinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; progressive supranucleo palsy; cerebellar and spinocerebellar disorders, such as astructural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and Machado Joseph)); and systemic disorders (Refsum's disease, abetalipoprotemia, ataxia, telangiectasia, and mitochondrial multi-system disorder); demyelinating core disorders, such as multiple sclerosis, acute transverse myelitis; disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; senile dementia of Lewy body type; Wernicke-Korsakoff syndrome; chronic alcoholism; Creutzfeldt-Jakob disease; subacute sclerosing panencephalitis, Hallerrorden-Spatz disease; and Dementia pugilistica, or any subset thereof.

It is also recognized that malignant pathologies involving tumors or other malignancies, such as, but not limited to leukemias (acute, chronic myelocytic, chronic lymphocytic and/or myclodyspastic syndrome); lymphomas (Hodgkin's and non-Hodgkin's lymphomas, such as malignant lymphomas (Burkitt's lymphoma or mycosis fungoides)); carcinomas (such as colon carcinoma) and metastases thereof; cancer-related angiogenesis; infantile hemangiomas; and alcohol-induced hepatitis. Ocular neovascularization, psoriasis, duodenal ulcers, angiogenesis of the female reproductive tract, can also be treated when demonstrated by the diagnostic procedures described herein to be associated with chlamydial infection.

Ear Infections

Ear infections typically affect the middle or the external ear and include, for example, otitis media, otitis externa, and infections caused by surgical interventions. Due to multiplicity of secondary complications that arise from ear infections such as hearing loss, the treatment and prevention of such conditions is critical.

Topical administration of a metal complex or formulation of the invention is effective in treating or preventing an infection of the ear, such as otitis media or otitis externa. In the case of otitis media or externa, infections are primarily caused by H. influenza, M. catarhalis, S. pneumoniae, S. pyogenes, S. intermedius, S. epidermidis, S. aureus, S. caprae, S. auriculis, S. capitis, S. haemolytis, P. aeroginosa, P. mirabilis, P. vulgaris, E. faecalis, or E. coli. A metal complex or formulation of the invention can be used to treat each of these infections of the ear. The metal complex or formulation may, for example, be topically administered to the area of the ear to which surgical intervention was performed or, alternatively, the rifamycin may be administered to the ear of the patient prophylactically, prior to otic surgery, noninvasive otic procedures, or other types of surgery. Exemplary surgical procedures include for example, cochlear implant surgery, tympanoplasty, tympanostomy tube insertion, removal of tumors (e.g., cholesteatoma), or stapedectomy. The compound may be administered to the area of the ear to which surgical intervention will be performed, for example, within seven days, two days, one day, 12 hours, 10 hours, 6 hours, 4 hours, 2 hours, 1 hour, or less than 1 hour prior to or following the surgical intervention. The compositions may be used for acute treatment of temporary conditions, or may be administered chronically.

A metal complex or formulation of the invention may be given daily (e.g., once, twice, three times, or four times daily) or less frequently (e.g., once every other day, or once or twice weekly). Typically, patients are administered a dosage consisting of one to four drops of solution. The metal complex or formulation may be contained in any appropriate amount in any suitable carrier substance, and is generally present in an amount between 0.001% and 5%, desirably 0.01% and 3%, more desirably 0.1% and 1%, and even more desirably 0.1% and 0.4% by weight of the total volume (w/v) of the composition. The compound is provided in a dosage form that is suitable for topical administration. Thus, a composition containing a metal complex or formulation of the invention may be in the form of a solution, aerosol, gel, ointment, nebulizer, or suspension. Alternatively, the rifamycin may be administered by placing an impregnated porous media into the external ear canal to the tympanic membrane. The pharmaceutical composition can generally be formulated according to conventional pharmaceutical practice.

Aural Toilet

The external auditory canal and tissues lateral to the infected middle ear often are covered with mucoid exudate or desquamated epithelium. Since topically applied preparations cannot generally penetrate affected tissues until these interposing materials are removed, aural toilet is desirably performed before administering a metal complex or formulation of the invention. Aural toilet may be performed by a health provider, the patient, or any other individual. Removal of debris may be performed mechanically with the assistance of a microscope and microinstruments. Aural irrigation may also be performed using a solution containing peroxide. The concentration of peroxide should be the highest concentration without causing significant pain, or discomfort, to the patient. As an example, a solution of 50% peroxide and 50% sterile water can be used. Thirty to 40 mL of this solution can be irrigated through the external auditory canal, using a small syringe or bulb-type aspirator. The irrigant solution is allowed to drain out (e.g., for 5-10 minutes) prior to administering a rifamycin of the present invention.

Granulation Tissue

Granulation tissue often fills the middle ear and medial portions of the external auditory canal, and reducing this accumulation is beneficial for resolution of an ear infection. Granulation tissue may also prevent topically applied antimicrobial agents from penetrating to the site of infection, and the amount of granulation tissue is desirably reduced throughout the regimen. Although topical antimicrobial drops can reduce granulation by eliminating infection and by removing the inciting irritating inflammation, the amount of granulation tissue may be reduced using other methods known in the art. For example, topical steroids may hasten the resolution of middle ear granulation, thus improving penetration of topically delivered antibiotics.

Cautery may also be used to reduce the amount of granulation tissue and to reduce its formation. Microbipolar cautery may be administered by a health provider. Chemical cautery, using for example silver nitrate, may also be applied to an infected ear in the form of silver nitrate sticks. Excision of granulation tissue may also be performed by a health care provider with a microscope and microinstruments.

Ear Canal Acidification

In a patient affected with otitis extema, a therapy involving ear canal acidification to restore the physiological acidity of the ear may be performed. The affected ear is administered with a solution containing acetic acid, which may also include a steroid (e.g., hydrocortisone), aluminum acetate, or rubbing alcohol.

Topical Formulations

Pharmaceutical compositions according to the present invention can be formulated for topical administration to the ear of the patient. Patients having an ear infection may be administered with effective amounts of the metal complex or formulation of the invention, by means of a solution (e.g., drops), ointment, gel, or aerosol (e.g., nebulizer). The composition is typically administered to the affected otic area by topically applying, for example, one to four drops of a solution or suspension, or a comparable amount of an ointment, gel, or other solid or semisolid composition, once, twice, three times, or more than three times per day. A porous media or an ear wick (e.g., cotton, gauze, or compressed hydroxycellulose) may also be used to increase the penetration of the metal complex or formulation to the infected otic area. The ear wick, which is inserted into the canal under direct vision, is typically a dried sponge that helps wick eardrops along the canal, hold the solution in contact with the skin of the canal and apply pressure to the canal skin. Wicks may be removed at one day, two days, or more than two days, and may be replaced if necessary. Alternatively, the ear wick may itself be impregnated with the metal complex or formulation. These formulations can be made according to known and conventional methods for preparing such formulations.

Since some of the metal complexes or formulations of the invention of the invention may not be highly soluble in water at physiological conditions, a solubilizing excipient may be used to increase solubility. Solubilization is taken to mean an improvement in the solubility by virtue of surface-active compounds that can convert substances that are insoluble or virtually insoluble in water into clear, or opalescent, aqueous solutions without changing the chemical structure of these substances in the process. Excipients used for this purpose are restricted to those that are safe for administration to humans. Typically such co-solvents are employed at a level of about 0.01% to 2% by weight.

A variety of solubilizing excipients may be used for formulation, including compounds belonging to the following classes: polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, or ionic surfactants. Such excipients are described for example, in U.S. patent application Ser. No. 60/385,532, hereby incorporated by reference.

Ototopical preparations may vary in viscosity. The use of viscosity enhancing agents to provide the compositions of the invention with viscosities greater than the viscosity of simple aqueous solutions may be desirable to increase the retention time in the ear. Such viscosity-building agents include, for example, polyvinyl alcohol, polyvinyl pyrrolidone, methyl cellulose, hydroxypropyl methylcellulose, hydroxyethyl cellulose, carboxymethyl cellulose, hydroxypropyl cellulose, or other agents known to those skilled in the art. Such agents are typically employed at a level of about 0.01% to 2% by weight. Optionally, these preparations may include a buffering agent to maintain an acidic pH, since the normal environment of the external auditory canal is acidic. However, if treatment is required in the middle ear where the pH is neutral, the pH can be adjusted accordingly.

Otic pharmaceutical products are typically packaged in multidose form. Preservatives are thus desired to prevent microbial contamination during use. Suitable preservatives include: polyquaternium-1, benzalkonium chloride, thimerosal, chlorobutanol, methyl paraben, propyl paraben, phenylethyl alcohol, edetate disodium, sorbic acid, or other agents known to those skilled in the art. Typically such preservatives are employed at a level of from 0.001% to 1.0% by weight.

A penetration enhancer may also be used to facilitate the diffusion of the metal complex or formulation through the tympanic membrane into the middle and inner ear in order to reduce inflammation of ear tissues. A penetration enhancer is an agent used to increase the permeability of the skin to a pharmacologically active agent to increase the rate at which the drug diffuses through the skin and enters the tissues and bloodstream. A chemical skin penetration enhancer increases skin permeability by reversibly damaging or by altering the physiochemical nature of the stratum corneum to reduce its diffusional resistance (Osborne D W, Henke J J, Pharmaceutical Technology, November 1997, pp 58-86). Examples of penetration enhancers include without limitation: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and admixtures thereof The use of such penetration enhancers is disclosed, for example, in U.S. Pat. No. 6,093,417, hereby incorporated by reference.

Other Therapeutic Agents

Preparations containing a metal complex or formulation of the invention may also include a second therapeutic agent, including for example, another rifamycin, an anesthetic, an antimicrobial agent, a zinc salt, or an anti-inflammatory agent (e.g., an non-steroidal anti-inflammatory or a steroid). When admixing an antimicrobial agent, the antimicrobial agent is preferably amoxillin, erythromycin, azithromycin, clarithromycin, gentamicin, tobramycin, ciprofloxaxin, norfloxacin, gatifloxacin, ofloxacin, levofloxacin, moxifloxacin, metronidazole, lomefloxacin, ciprofloxacin, natamycin, neomycin, polymyxin B, gentamycin, bacitracin, trovafloxacin, grepafloxacin, sulfacetamide, tetracycline, gramicidin, chloremphenicol, or gramicidin. Preferred non-steroidal anti-inflammatory agents include, for example, detoprofen, diclofenac, diflunisal, etodolac, fenoprofen, flurbiprofen, indomethacin, ketoprofen, mechlofenameate, mefenamic acid, meloxicam, nabumeone, naproxen sodium, oxaprozin, piroxicam, sulindac, tolmeting, celecoxib, rofecoxib, choline salicylate, salsate, sodium salicylate, magnesium salicylate, aspirin, ibuprofen, paracetamol, acetaminophen, and pseudoephedrine, and preferred steroids include, for example, hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone. Preferred anesthetics according to the invention include, for example, benzocaine, butamben picrate, tetracaine, dibucaine, prilocaine, etidocaine, mepivacaine, bupivicaine, and lidocaine. A zinc salt can be zinc sulfate, zinc chloride, zinc acetate, zinc phenol sulfonate, zinc borate, zinc bromide, zinc nitrate, zinc glycerophosphate, zinc benzoate, zinc carbonate, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, or zinc glycinate. All of the therapeutic agents employed in the compositions of the present invention can be used in the dose ranges currently known and used for these agents. Different concentrations may be employed depending on the clinical condition of the patient, the goal of therapy (treatment or prophylaxis), the anticipated duration, and the severity of the infection for which a metal complex or formulation of the invention of the invention is being administered. Additional considerations in dose selection include the type of infection, age of the patient (e.g., pediatric, adult, or geriatric), general health, and comorbidity.

Therapeutic compositions may be in the form of liquid solutions or suspensions; for oral administration, compositions may be in the form of tablets or capsules; and for intranasal formulations, in the form of powders, nasal drops, or aerosols.

Formulations of rifamycin analogues of formula I and a biocompatible metal salt can be prepared in any manner known to those skilled in the art of pharmaceutical formulations. In one example, formulations of the present invention can be made in a manner similar to that described by U.S. Pat. No. 5,547,683 (hereby incorporated by reference). Further details are provided in Example 1. Methods well known in the art for making formulations are found, for example, in Remington: The Science and Practice of Pharmacy (20th ed., ed. A. R. Gennaro A R.), Lippincott Williams & Wilkins, 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the metal complex or formulation of rifamycin analogue plus metal salt in the formulation will vary depending upon a number of factors, including the dosage of the drug to be administered, and the route of administration.

The metal complex or formulation may be optionally administered with an additional pharmaceutically acceptable salt, such as non-toxic acid addition salts that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose; and inorganic acid such as hydrochloric acid, hydrobromic acid, sulfuric acid phosphoric acid.

The metal complexes and formulations for use in such therapies may be produced and isolated as described herein or by any standard technique known to those in the field of medicinal chemistry. Conventional pharmaceutical practice may be employed to provide suitable compositions to administer the identified complex or formulation to patients suffering from a condition or at increased risk for a condition involving bacterial infection. Administration may begin before or after the patient is symptomatic.

The complex or formulation can be administered to human patients in therapeutically effective amounts (e.g., amounts which prevent, stabilize, eliminate, or reduce a bacterial infection) to provide therapy for a disease or condition associated with a bacterial infection. Typical dose ranges are from about 0.1 μg/kg to about 1 mg/kg of body weight per day. The exemplary dosage of drug to be administered is likely to depend on such variables as the type and extent of the disorder, the overall health status of the particular patient, the formulation excipients, and its route of administration. Standard clinical trials maybe used to optimize the dose and dosing frequency for any particular complex or formulation of the present invention.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLE 1

### Metal Salt-Rifamycin Analogue Formulations

To produce metal salt-rifamycin analogue formulations, a fine powder having the average particle size of not more than 10 μm is obtained by pulverizing a given compound of formulas I, a metal salt, and a carrier.

The carrier for obtaining such a fine powder is not subject to limitation; usually, the most effective carrier is selected from the group of talc, calcium hydrogen phosphate, silicic anhydride, crystalline cellulose, lactose, mannitol, etc. As for the apparatus for pulverizing, it is useful to employ a mill, such as a jet mill or a hammer mill. Using such a mill, the subject compound is finely powdered to a particle size of not more than 10 μm. Some starting materials do not need a carrier. Often, the resulting milled powder is a suitable drug substance.

In some cases, modifications to the particle surface will be desirable. The above fine powder can be further granulated in the presence of a binder such as a water-soluble polymer (e.g., starch glue, carmellose, tragacanth, gum arabic, sodium alginate, hydroxypropyl cellulose, hydroxypropyl methyl cellulose, sodium carboxymethyl cellulose and polyvinylpyrrolidone) or in the presence of such a binder and a surfactant (e.g., sodium dodecyl sulfate), which is combined with a surface treatment using particles smaller than the fine powder as a fluidizing agent to accomplish microgranulation giving a very uniform particle size distribution. In the granulation a binder or a binder and a surfactant are sprayed via respective nozzles onto the surface of the fine powder being agitated, tumbled, or fluidized. In this spraying, the amount of surfactant added is normally not more than 10% by weight of the fine powder, and the amount of water-soluble polymer added is normally 1 to 20% by weight of the fine powder.

For the surface treatment performed by mixing and stirring fine particles in the present invention, particles smaller than the fine powder can be used, and high speed dry mixing, for example, can be used. In the present invention, by mixing and stirring such fine particles, they are made to adhere to the surface of the fine powder or made to be co-present in the vicinity of the surface.

An apparatus usable for both the process of mixing and stirring a fine particle and the process of microgranulation in the present invention should have multiple functions as described below, in addition to an ordinary function of fluidized bed granulation coating. An example is Wurster fluidized bed granulation coaters (e.g., those produced by Glatt K. K. or Powrex Corporation). This apparatus, which has a cylindrical Wurster column set at the center of a container, can fluidize a fine powder or a granulated particle through the column in a single direction by an upward gas stream (jet stream), spray fine droplets of a binder or those of a binder and a surfactant to the subject particle from the jet nozzle at the bottom for coating (bottom spray method) and perform granulation and drying.

In addition to the above-described apparatus, multi-function combined granulation coaters of the agitating tumbling fluidized bed type (e.g., SPIR-A-FLOW granulation coater, produced by Freund Industrial Co., Ltd., and New-Marumerizer, produced by Fuji Paudal Co., Ltd.), multi-function combined granulation coaters of the tumbling fluidized bed type (e.g., Multiplex, produced by Powrex Corporation) and other apparatuses can also be used. Spraying methods of these multi-function combined granulation coaters include the top spraying method, in which droplets are sprayed from the top, the middle spraying (tangential spraying) method, in which droplets are sprayed from a side of the bottom, and the bottom spraying method. However, the middle spraying (tangential spraying) method or the bottom spraying method is effective for microgranulation in many cases. In short, it is necessary to control the granulation to yield a microgranulated particle by binding together uniformly coated particles, which can be accomplished by preventing flocking (aggregation) of the subject particles during the granulation process by minimizing the diameter of the droplets of a binder and a surfactant and increasing the speed at which the droplets collide with the fine powder or granulated particle during spraying and granulation. Such apparatus with multiple functions is exemplified by the one using one of the above-described spraying methods. The concentration/amount of the fine particle, hydrophilic surfactant, binder, etc. used for such surface treatment and granulation are optionally chosen according to the apparatus used so that the granulated particle has desired particle size of not more than 0.2 mm. The particle size mentioned here is the measurement obtained by scanning electron microscopy or by sieving.

The granulated particle thus obtained has a particle size of not more than 0.2 mm, preferably not more than 0.1 mm, very sharp particle size distribution, improved fluidity and improved water wetting.

### EXAMPLE 2

### Metal-Rifamycin Analogue Complexes

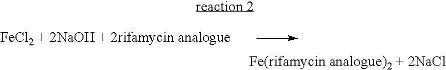

To generate metal-rifamycin analogue complexes, anhydrous ferrous chloride (Aldrich catalogue number 42,936-8), 0.126 g, 1 mmol, is dissolved in 40 mL absolute ethanol. To the iron solution is added, dropwise, two equivalents of MTI-200 from a stock solution in absolute ethanol, as shown in reaction 2. After stirring for 20 minutes at room temperature, two equivalents of sodium hydroxide are added dropwise to the reaction mixture until the solution is neutralized (pH may be between 5-8). Following an additional 20 minutes of stirring, the volatiles are removed under vacuum. The resulting solid is suspended in a minimum amount of cooled water, isolated on a filter, rinsed twice with water, and dried.

The details of a typical synthesis are provided above. In cases where the metal-rifamycin analogue complex is soluble in aqueous solution, the resulting solid may be dissolved in water and desalted by reverse phase chromatography, followed by lyophilization of the purified aqueous complex.

Complexes with other metals can be prepared in an analogous manner.

### EXAMPLE 3

### Synthesis of Sulfhydryl Benzoxazinorifamycin Analogues

Rifamycin analogues can be prepared using methods which require the selective protection and deprotection of alcohols, amines, sulflhydryls and/or carboxylic acid functional groups. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxylic acids include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxylic acid functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994 (hereby incorporated by reference). In the examples that follow, the use of protecting groups is indicated in a structure by the letter P, where P for any amine, carboxylic acid, sulfhydryl, or alcohol may be any of the protecting groups listed above.

Precursor Sulfhydryl Amino Phenol

Rifamycin derivatives having the formula I in which R² is sulfhydryl may be prepared by reacting rifamycin S (LKT Laboratories, Inc., catalogue number DR32202) with a compound having the formula IV.

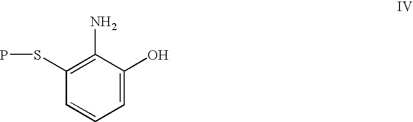

Compounds of formula IV are prepared from 2-aminoresorcinol (Chem Service, Inc., catalogue number 1895B) as shown in reaction 1. The unprotected hydroxyl can be activated using standard techniques (e.g., conversion to a tosylate,

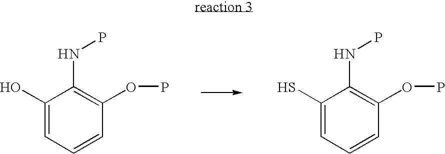

brosylate, mesylate, triflate or other reactive leaving group see, for example, J. March, Advanced Organic Chemistry: Reactions, Mechanisms and Structure, John Wiley & Sons, Inc. pp. 352-354, 1992). The conversion of the activated alcohol to a sulfhydryl group can be achieved by either addition of sulfide (e.g., NaSH, Na₂S), addition of disulfide (e.g., Na₂S₂) followed by reduction of the disulfide to a sulfhydryl group, or transesterification of the activated alcohol with thioacetate followed by hydrolysis to the sulfhydryl with sodium acetate. The reaction product is converted into a compound of formula IV using standard protection and deprotection chemistry.

Synthesis of Sulfhydryl Rifamycin

In a typical reaction, a compound of formula IV is added in small portions to a solution of rifamycin S in chloroform containing several equivalents of triethylamine. This can be followed by the addition of manganese dioxide and the reaction stirred until reaching completion. The resulting compound will have formula V shown below.

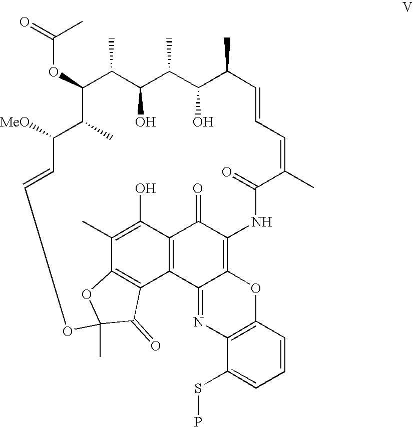

Synthesis of Sulfhydryl Rifamycin Analogues of Formula I

The sulfhydryl rifamycin product is further modified, using the methods disclosed in U.S. Pat. No. 4,690,919. A compound represented by formula IV is dissolved in DMSO, mixed with N-isobutylpiperazine and manganese dioxide, and the reaction mixture stirred at room temperature for 3 hours. The resulting product is shown below.

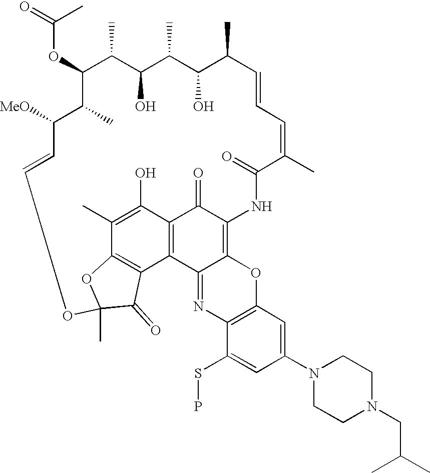

The sulfhydryl protecting group can be removed, resulting in a compound of formula I.

Compounds in which R³ of formula I is selected from other groups can be prepared by an analogous method.

Synthesis of Compounds of Formula I in which X¹ is Sulfur

Compounds for which X¹ of formula I is a sulfur atom are prepared by an analogous method, but using a starting material of formulas VI or VII, shown below.

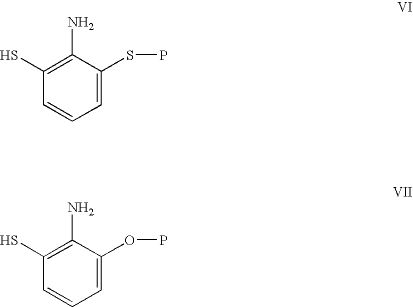

These materials can also be prepared from 2-aminoresorcinol. For example, a compound of formula V can be prepared by converting both hydroxyls of 2-aminoresorcinol to sulfhydryls, vide supra, followed by the deprotection and/or protection of functional groups.

A compound of formulas VI or VII can be combined with rifamycin S, vide supra, to produce the sulfhydryl rifamycin intermediate of formulas VIII or IX, respectively, shown below.

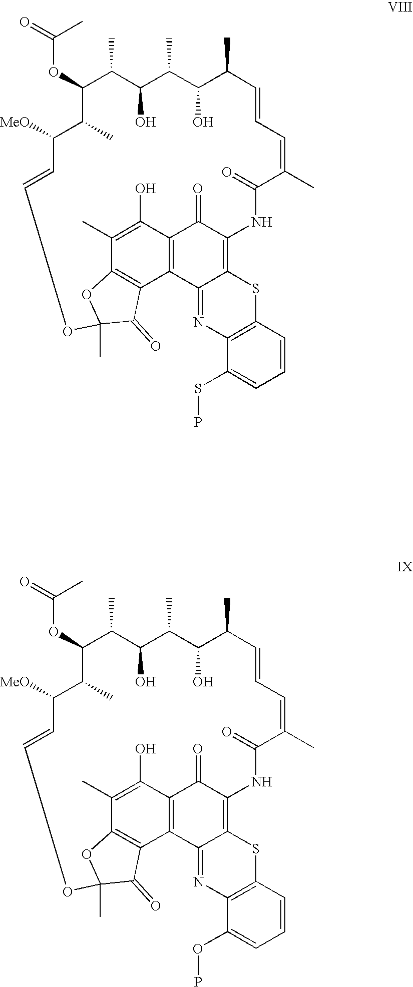

To the intermediate of formula VI may be added R³, as defined in formula I, using the methods disclosed in U.S. Pat. No. 4,690,919, and described herein.

### EXAMPLE 4

### MICAssay

MICs of candidate compounds of the invention can be determined, for example, by the method of Lee et al., Am. Rev. Respir. Dis. 136:349 1987. To a BACTEC 12B vial (4 mL of 7H12B medium), 0.1 mL of a 10-fold dilution of subculture of the test organisms in 7H9 medium (optical density at 540 nm, 0.1) is inoculated and cultured at 37° C. until a growth index (GI) of 999 is reached. Then the broth culture is removed and diluted 100-fold, and 0.1 mL of the dilution is inoculated into a BACTEC 12B vial with or without a candidate compound. The candidate compound containing vials can hold 0.1 mL of candidate compound solution appropriately diluted to obtain the desired concentration. A 1% control vial, 0.1 ml of the 100-fold dilution of the inoculum described above, is inoculated into 12B vial without candidate compound. The 12B vials are incubated at 37° C., and GI readings recorded daily, using a BACTEC 460 TB instrument (Johnston Laboratories, Townsend, Md.), until the control vial reaches a GI greater than 30. When the final readings in the GI of the candidate containing vials are lower than those of the 1% control, the drug is considered to have inhibited more than 99% of the bacterial population, and this concentration is defined as the MIC.

### EXAMPLE 5

### Synthesis of Compound 7, a Sulfhydryl Rifamycin Derivative

Synthesis of Compound 2

Compound 1 (9.95 g, 59.9 mmol) was added to an aqueous solution of KOH (200 mL, 9 M). The reaction was stirred at reflux for 22 hours and then cooled to room temperature. The reaction was extracted once with ether, followed by acidification with 50% acetic acid (pH 5-6). The aqueous layer was extracted with methylene chloride (×5), and the combined organics were dried over Na₂SO₄. Filtration followed by removal of the solvent in vacuo yielded a mixture of the free thiol and 2 as a yellow solid (7.82 g) that was used with out further purification.

To a stirred solution of the above mixture (7.79 g) in acetonitrile (280 mL) and water (140 mL) was bubbled air for 32 hours. The reaction was poured over water and the aqueous layer was extracted with methylene chloride (×5). The combined organics were dried over Na₂SO₄ and filtered. Removal of the solvent in vacuo yielded the title compound 2 as a light-brown solid (7.46 g, 26.6 mmol, 89%, 2-step), which was used with out further purification. ¹H NMR (CD₃OD, 300 MHz): 6.38-6.44 (m, 2H), 6.66-6.72 (m, 4H). ESI (+) MS: 281 (M+H⁺).

Synthesis of Compound 3

To a stirred solution of 2 (2.33 g, 8.31 mmol) in THF (40 mL) was added di-tert-butyl dicarbonate (7.25 g, 33.2 mmol). The reaction was stirred at 50° C. for 20 hours and then cooled to room temperature. The solvent was removed in vacuo, and the resulting residue was dissolved in ethyl acetate, washed with KHSO₄ (0.5 M), water, brine and dried over Na₂SO₄. The solution was decanted, and the solvent removed in vacuo. The resulting residue was purified via flash chromatography (silica gel, methylene chloride) to yield the title compound 3 as a yellow-brown viscous oil (3.60 g, 7.49 mmol, 90%). ¹H NMR (CDCl₃, 300 MHz): 1.55 (s, 18H), 4.39 (s, 4H), 6.58 (t, j=7.8 Hz, 2H), 7.06-7.11 (m, 4H). ESI (+) MS: 481 (M+H⁺).

Synthesis of Compound 4

To a stirred solution of 3 (1.07 g, 2.23 mmol) in DMF (11 mL, sparged with N₂) was added water (41 μL, 2.27 mmol) and tri-n-butylphosphine (835 μL, 3.35 mmol). The reaction was stirred at room temperature under N₂ for 1.5 hours. 3-chloropropionitrile (350 μL, 4.46 mmol) and K₂CO₃ (616 mg, 4.46 mmol) were added, and the reaction was stirred at room temperature under N₂ for 14.5 hours. The reaction was diluted with ethyl acetate, filtered and poured over water. The organic layer was washed with water and 5% LiCl (×2), dried over Na₂SO₄, filtered and the solvent removed in vacuo. The resulting residue was purified using MPLC (1:1, Ethyl acetate:Hexanes) and repurified via flash chromatography (silica gel, 1:1, Ethyl acetate:Hexanes) to yield the title compound 4 as a colorless oil (534 mg, 1.81 mmol, 41%). ¹H NMR (CDCl₃, 300 MHz): 1.56 (s, 9H), 2.53 (t, j=6.9 Hz, 2H), 2.96 (t, j=6.9Hz, 2H), 4.50 (s, 2H), 6.70 (t, j=7.8 Hz, 1H), 7.14 (dd, j=1.3, 8.0 Hz, 1H), 7.29 (dd, j=1.3, 7.7 Hz, 1H). ESI (+) MS: 239 (M−tBu⁺+2H+), 195 (M−Boc⁺+2H⁺).

Synthesis of Compound 5

A solution of 4 (523 mg, 1.78 mmol) in methylene chloride (5 mL) and trifluoroacetic acid (5 mL) was stirred at room temperature for 2.5 hours. The solvent was removed in vacuo, and the resulting residue was dissolved in methylene chloride and washed with NaHCO₃ solution. The aqueous layer was extracted with methylene chloride and the combined organics were dried over Na₂SO₄. Filtration, followed by removal of the solvent in vacuo yielded the free amino-phenol as a white solid that was used without further purification.

A solution of the above amino-phenol and Rifamycin S (1.24 g, 1.78 mmol) in toluene (15 mL) was stirred at 50° C. under N₂ for 18 hours, and at 60° C. for an additional 8 hours. The reaction was cooled to room temperature and the solvent was removed in vacuo. The resulting residue was dissolved in EtOH (15 mL), and to this was added manganese dioxide (1.55 g, 17.8 mmol). The suspension was stirred at room temperature for 22 hours. The reaction was filtered through Celite, and the solvent removed in vacuo. The resulting residue was purified via MPLC (7:3, Ethyl acetate:Hexanes) to yield the title compound 5 as a red solid (656 mg, 0.754 mmol, 42%). ESI (+) MS: 838 (M−OMe⁻), 870 (M+H⁺), 892 (M+Na⁺). TLC: R_{f}=0.52 (1:9, methylene chloride: ethyl acetate, Whatman MK6F, 60 angstrom).

Synthesis of Compound 6

To a stirred solution of 5 (550 mg, 0.632 mmol) in methyl sulfoxide (10 mL) was added MnO₂ (549 mg, 6.32 mmol) and 1-isobutylpiperazine (90 mg, 0.632 mmol), and the resulting suspension was stirred at room temperature for 39 hours. The reaction was diluted with ethyl acetate and filtered through Celite. The resulting solution was washed with water (×4) then brine, and dried over Na₂SO₄. Filtration, followed by removal of the solvent in vacuo yielded a blue residue that was purified via flash chromatography (silica gel, 9:1, ethyl acetate:methylene chloride). Impure material was further purified via MPLC (1:39, methanol:methylene chloride). All pure materials were pooled, and the solvent removed in vacuo to yield the title compound 6 as a blue solid (165 mg, 0.163 mmol, 26%). ESI (+) MS: 1010 (M+H⁺). TLC: R_{f}=0.34 (1:9, methylene chloride: ethyl acetate, Whatman MK6F, 60 angstrom).

Synthesis of Compound 7

To a stirred solution of 6 (14 mg, 13.9 μmol) in THF (1 mL) at 0° C. was added potassium tert-butoxide (8 mg, 6.95 μmol). The reaction was stirred at 0° C. for 10 minutes. Acetic acid was added dropwise until the solution turned from green to blue. The reaction was diluted with ethyl acetate and washed with NH₄Cl solution and water. The organics were dried over Na₂SO₄, decanted, and the solvent removed in vacuo. The resulting residue was purified via preparatory TLC (1:9 methanol:methylene chloride) to yield compound 7 as a blue film (6 mg, 6.27 μmol, 45%). ESI (+) MS: 957 (M+H⁺). UV/Vis: λₘₐₓ−582.7 nm

### Example 6

### Compound 8—Fe(II) Complex

To a solution of 6.00 g (6.38 mmol) of of compound 8 (rifalazil) in 400 mL of methanol was added 0.808 g (6.38 mmol) of Iron (II) chloride. The mixture was stirred for three hours at room temperature. The reaction was monitored by UV-Vis (200-800 nm). The solvent was evaporated on air and dried in vacuo at room temperature for six hours to obtain the resulting product (6.9 g). The solubility in water for the resulting ferrous complex of rifalazil is 86.4 mg/mL. UV/Vis: λₘₐₓ=618.0 nm, 354.5 nm, 270.0 nm, 216.5 nm, 209.5 nm. ESI (+) MS: 996 (Fe-rifalazil+H⁺), 1030.5 (FeCl−rifalazil+H⁺).

### Other Embodiments

All publications and patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims. What we claim is:

1. A composition comprising a rifamycin analogue and a metal salt, wherein the metal salt is added in an amount sufficient to reduce the minimum inhibitory concentration of the rifamycin analogue, and the rifamycin analogue is a compound of formula I: 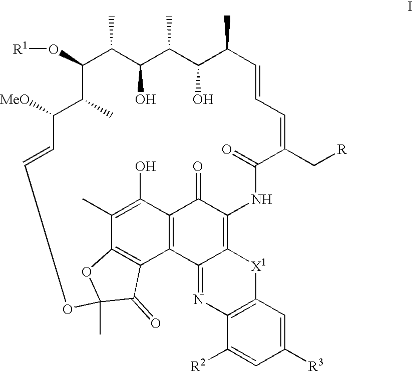
wherein
X¹ represents an oxygen atom or a sulfur atom;
R represents a hydrogen atom or hydroxyl group;
R¹ represents acetyl or H, OH;
R² represents a hydroxyl group or a sulfhydryl group; and
R represents: 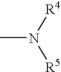
wherein each of R⁴ and R⁵ is, independently, an alkyl group having 1 to 7 carbon atoms, or R⁴ and R⁵ combine to form a 3-8 membered cyclic system,
or R³ represents a group expressed by the formula: 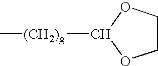
in which g represents an integer between 1 and 3,
or R³ represents a group expressed by the formula: 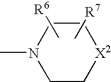
wherein each of R⁶ and R⁷ is, independently, a hydrogen atom or an alkyl group having 1 to 3 carbon atoms, X² represents an oxygen atom, a sulfur atom, a carbonyl group,
or X² represents: 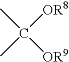
in which each of R⁸ and R⁹ is, independently, a hydrogen atom, or an alkyl group having 1 to 3 carbon atoms, or R⁸ and R⁹, in combination with each other, represent —(CH₂)k— in which k represents an integer between 1 and 4,
or X² represents: 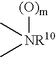
in which m represents 0 or 1, R¹⁰ represents a hydrogen atom, an alkyl group having 1 to 6 carbon atoms, or —(CH₂)ₙX³ in which n represents an integer between 1 and 4, and X³ represents an alkoxy group having 1 to 3 carbon atoms, a vinyl group, an ethynyl group,
or R¹⁰ represents:

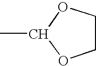

2. The composition of claim 1, wherein said rifamycin analogue is described by formula II: 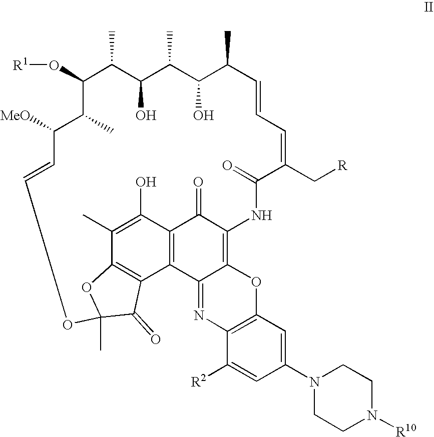
wherein
R represents a hydrogen or a hydroxyl group;
R¹ represents hydrogen or an acetyl group;
R² is hydroxyl or sulfhydryl; and
R¹⁰ is selected from the group consisting of methyl, ethyl, iso-propyl, n-propyl, iso-butyl, (S)-sec-butyl, and (R)-sec-butyl.

3. The composition of claim 2, wherein said rifamycin analogue is described by the chemical structure:

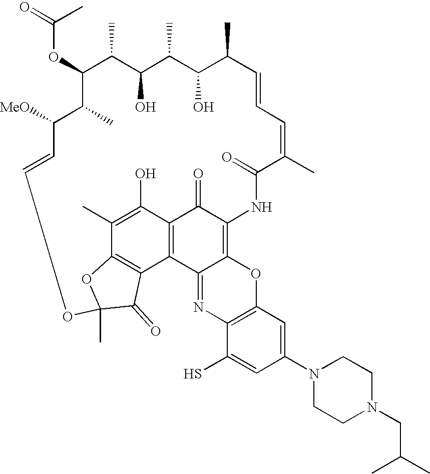

4. The composition of claim 1, wherein said metal salt comprises metals selected from the group consisting of Groups I (A, B), II (A, B), III (A, B), IV(A, B), V(A, B), VIA, VIIA, VIII, and combinations thereof.

5. The composition of claim 1, wherein said metal salt comprises one or more metals selected from the group consisting of zinc, iron, copper, ruthenium, gallium, aluminum, nickel, cobalt, and combinations thereof.

6. The composition of claim 5, wherein said metal salt is an iron salt.

7. The composition of claim 5, wherein the mole ratio of metal to rifamycin analogue in the composition falls within the range of 0.1 to 10.

8. The composition of claim 5, wherein in a Chlamydia growth assay the minimum inhibitory concentration of the rifamycin analogue formulated with a metal salt is less than 50% of the minimum inhibitory concentration of the rifamycin analogue formulated without a metal salt.

9. A method of preventing, stabilizing, or inhibiting the growth of bacteria, or killing bacteria, said method comprising contacting bacteria or a site susceptible to bacterial growth with a composition of claim 1.

10. The method of claim 9, wherein the step of contacting bacteria or a site susceptible to bacterial growth comprises administering to the animal a composition of claim 1 in an amount sufficient to treat or prevent the bacterial infection.

11. A metal-rifamycin analogue complex comprising a metal selected from the group consisting of Groups I (A, B), II (A, B), III (A, B), IV (A, B), V (A, B), VIA, VIIA, and VIII; and a rifamycin analogue of formulas I or II.

12. The metal-rifamycin analogue complex of claim 11, wherein said metal-rifamycin analogue complex comprises one or more metals selected from the group consisting of zinc, iron, copper, ruthenium, gallium, aluminium, nickel, cobalt, and combinations thereof.

13. The metal-rifamycin analogue complex of claim 11, wherein said complex is described by formula III:
Mₐ(rifamycin analogue)_{b} III
in which M is a metal, rifamycin analogue is a compound of formula I, and a and b each, independently, represent an integer from 1 to 10, inclusive.

14. The metal-rifamycin analogue complex of claim 11, wherein said metal-rifamycin analogue complex contains iron in an oxidation state selected from the group consisting of +4, +3, +2, +1, and combinations thereof.

15. A metal-rifamycin analogue complex comprising a metal selected from the group consisting of zinc, iron, copper, ruthenium, gallium, aluminium, nickel, cobalt, and combinations thereof; and a rifamycin analogue of formula II: 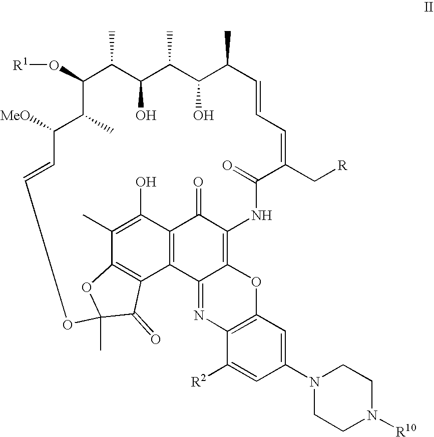
wherein
R represents a hydrogen or a hydroxyl group;
R¹ represents hydrogen or an acetyl group;
R² is hydroxyl or sulfhydryl; and
R¹⁰ is selected from the group consisting of methyl, ethyl, iso-propyl, n-propyl, iso-butyl, (S)-sec-butyl, and (R)-sec-butyl.

16. The metal-rifamycin analogue complex of claim 15, wherein said rifamycin analogue is described by the chemical structure:

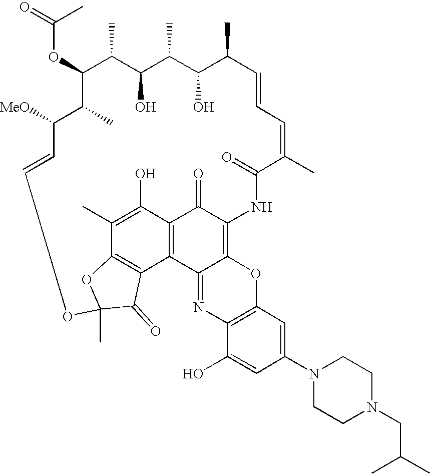

17. A method of preventing, stabilizing, or inhibiting the growth of bacteria, or killing bacteria, said method comprising contacting bacteria or a site susceptible to bacterial growth with a complex of claim 11 or 15.

18. The method of claim 17, wherein the step of contacting bacteria or a site susceptible to bacterial growth comprises administering to an animal a complex of claim 11 in an amount sufficient to treat or prevent the bacterial infection.

19. An aqueous solution comprising a metal-rifamycin analogue complex.

20. A pharmaceutical composition comprising the aqueous solution of claim 19 along with one or more pharmaceutically acceptable carriers or diluents.

21. The aqueous solution of claim 19, wherein the concentration of said metal-rifamycin analogue complex is between 0.10 and 200,000 μg/mL.

22. A method of treating or preventing disease in a human, said method comprising intravenous administration of the solution of claim 20 to said human in amounts effective to treat or prevent said disease.

23. The method of claim 22, wherein said intravenous administration comprises intravenous infusion into said human of between 2 and 50 mg of said metal-rifamycin analogue complex over a period of 4 to 24 hours.

24. The method of claim 23, wherein said intravenous administration comprises:
a) a bolus injection of between 2 and 25 mg of said metal-rifamycin analogue complex over 10 to 60 minutes, and
b) following step a, a slow infusion of between 0.1 and 2 mg per hour for up to 24 hours.

25. The method of claim 23, wherein said intravenous administration is repeated daily for a period of two to fourteen days.

26. A method of treating disease in a human, said method comprising intravenous administration of a metal-rifamycin analogue complex at a rate that maintains a plasma concentration of the rifamycin analogue, including both complexed and uncomplexed forms, of between 6 and 50 ng/mL in the plasma of said human for a period greater than 5 hours.

27. The method of 26, wherein the plasma concentration of said rifamycin analogue, including both complexed and uncomplexed forms, is between 10 and 30 ng/mL for a period greater than 24 hours.

28. A pharmaceutical composition comprising a pharmaceutically acceptable carrier or diluent and one or more of the following: a composition of claim 1; a metal complex of claim 11; a metal complex of claim 15; a metal and a racemic mixture of two or more compounds of formula I; a metal and two or more diastereomers of a compound of formula I; two or more metal-based structural isomers of formula III; two or more optical isomers of formula III; two or more diastereomers of formula III; or two or more complexes of formula III.

29. The composition of claim 28, further comprising a proton pump inhibitor or bismuth preparation.

30. The composition of claim 29, wherein said proton pump inhibitor is selected from the group consisting of omeprazole, esomeprazole, lansoprazole, leminoprazole, pantoprazole and robeprazole.

31. The composition of claim 29, wherein said bismuth preparation is selected from the group consisting of colloidal bismuth subcitrate and bismuth subsalicylate.

32. A method of killing, treating, or preventing a bacterial infection in an animal, said method comprising administering to the animal the pharmaceutical composition of claim 28.

33. The method of claim 32, wherein the bacterial infection is an intracellular infection.

34. The method of claim 33, wherein said intracellular bacterial infection is caused by an obligate intracellular bacterium.

35. The method of claim 34, wherein said bacterium is selected from the group consisting of Anaplasma bovis, A. caudatum, A. centrale, A. marginale A. ovis, A. phagocytophila, A. platys, Bartonella bacilliformis, B. clarridgeiae, B. elizabethae, B. henselae, B. henselae phage, B. quintana, B. taylorii, B. vinsonii, Borrelia afzelii, B. andersonii, B. anserina, B. bissettii, B. burgdorferi, B. crocidurae, B. garinii, B. hermsii, B. japonica, B. miyamotoi, B. parkeri, B. recurrentis, B. turdi, B. turicatae, B. valaisiana, Brucella abortus, B. melitensis, Chlamydia pneumoniae, C. psittaci, C. trachomatis, Cowdria ruminantium, Coxiella burnetii, Ehrlichia canis, E. chaffeensis, E. equi, E. ewingii, E. muris, E. phagocytophila, E. platys, E. risticii, E. ruminantium, E. sennetsu, Haemobartonella canis, H. felis, H. muris, Mycoplasma arthriditis, M. buccale, M. faucium, M. fermentans, M. genitalium, M. hominis, M. laidlawii, M. lipophilum, M. orale, M. penetrans, M. pirum, M. pneumoniae, M. salivarium, M. spermatophilum, Rickettsia australis, R. conorii, R. felis, R. helvetica, R. japonica, R. massiliae, R. montanensis, R. peacockii, R. prowazekii, R. rhipicephali, R. rickettsii, R. sibirica, and R. typhi.

36. The method of claim 32, wherein said animal is a human.

37. A method of treating a mammal having a condition caused by or contributed to by bacterial infection, said method comprising administering to said mammal a therapeutically effective amount of a composition of claim 28.

38. The method of claim 37 wherein said condition is selected from the group consisting of community-acquired pneumonia, upper and lower respiratory tract infections, skin and soft tissue infections, bone and joint infections and hospital-acquired lung infections.

39. The method of claim 37 wherein said infection is by a bacterium selected from the group consisting of S. aureus, S. epidermidis, S. pneumoniae, S. pyogenes, Enterococcus spp., M. catarrhalis, and H. influenzae.

40. The method of claim 37 wherein said infection is by a Gram-positive coccus.

41. The method of claim 40, wherein said Gram-positive coccus is drug-resistant.

42. A method of preventing a mammal from suffering from a condition caused by or contributed to by a bacterium, said method comprising administering to the subject a prophylactically effective amount of a composition of claim 28.

43. The method of claim 42, wherein said condition is selected from the group consisting of community acquired pneumonia, upper and lower respiratory tract infections, skin and soft tissue infections, bone and joint infections and hospital-acquired lung infections.

44. The method of claim 43, wherein said bacterium is selected from the group consisting of S. aureus, S. epidermidis, S. pneumoniae, S. pyogenes, Enterococcus spp., M. pneumoniae, M. catarrhalis, C. pneumoniae, K. pneumoniae, L. pneumophila, and H. influenzae.

45. The method of claim 43 wherein said bacterium is a Gram-positive coccus.

46. The method of claim 45 wherein said Gram-positive coccus is drug-resistant.

47. A method for treating or preventing the development of an atherosclerosis-associated disease in a patient in need thereof, said method comprising administering a composition of claim 28 to said patient in an amount effective to treat or prevent the development of said atherosclerosis-associated disease in said patient.

48. The method of claim 47, wherein the rifamycin analogue present in said composition is administered in an amount between 0.001 and 100 mg.

49. The method of claim 48, wherein said rifamycin analogue is administered in an amount between 1 and 50 mg.

50. The method of claim 49, wherein said rifamycin analogue is administered in an amount between 5 and 25 mg/week.

51. The method of claim 50, wherein said said rifamycin analogue is administered in an amount between 2.5 and 25 mg/day.

52. The method of claim 48, wherein said rifamycin analogue is administered at an initial does of 12.5 to 100 mg for one to seven consecutive days, followed by a maintenance dose of 0.005 to 10 mg once every one to seven days.

53. The method of claim 47, further comprising the step of administering to said patient an anti-inflammatory agent, antibacterial agent, platelet aggregation inhibitor, anticoagulant, antipyretic, or lipid lowering agent.

54. The method of claim 53, wherein said patient is administered an anti-inflammatory agent.

55. The method of claim 54, wherein said anti-inflammatory agent is ibuprofen, meloxicam, celecoxib, rofecoxib, aspirin, dexamethasone, methylprednisolone, prednisolone, or prednisone.

56. The method of claim 53, wherein said patient is administered an antibacterial agent.

57. The method of claim 56, wherein said antibacterial agent is azithromycin, clarithromycin, erythromycin, gatifloxacin, levofloxacin, amoxicillin, or metronidazole.

58. The method of claim 53, wherein said lipid lowering drug is a statin.

59. The method of claim 58, wherein said statin is atorvastatin, rosuvastatin, lovastatin, simvastatin, pravastatin, cerivastatin, or fluvastatin.

60. The method of claim 47, wherein said atherosclerosis-associated disease is coronary artery disease, myocardial infarction, angina pectoris, stroke, cerebral ischemia, intermittent claudication, gangrene, mesenteric ischemia, temporal arteritis, or renal artery stenosis.

61. The method of claim 47, wherein, prior to administration of said compound, said patient is diagnosed as having said atherosclerosis-associated disease.

62. A method of reducing the level of C-reactive protein in a patient identified as having increased levels of C-reactive protein, said method comprising administering a composition of claim 28 to said patient in an amount sufficient to reduce the level of C-reactive protein.

63. The method of claim 62, wherein said method further comprises the step of periodically monitoring the level of C-reactive protein in said patient following administration of said composition.

64. The method of claim 62, wherein the rifamycin analogue present in said composition is administered in an amount between 0.001 and 100 mg.

65. A method for reducing Chlamydia pneumoniae replication in macrophages or foam cells in a patient in need thereof, said method comprising administering a composition of claim 28 to said patient in an amount effective to reduce Chlamydia pneumoniae replication in macrophages or foam cells in said patient.

66. The method of claim 65, wherein the rifamycin analogue present in said composition is administered in an amount between 0.001 and 100 mg.

67. A method for treating a persistent Chlamydia pneumoniae infection in macrophages or foam cells in a patient, said method comprising administering a composition of claim 28 to said patient in an amount effective to treat said Chlamydia pneumoniae infection in macrophages or foam cells in said patient.

68. The method of claim 67, wherein the rifamycin analogue present in said composition is administered in an amount between 0.001 and 100 mg.

69. A method for treating or preventing a bacterial ear infection in a patient, said method comprising topically administering to the ear of said patient a composition of claim 28 in an amount effective to treat or prevent said ear infection in said patient.

70. The method of claim 69, wherein said patient is a human.

71. The method of claim 69, wherein said ear infection is otitis media.

72. The method of claim 71, wherein said otitis media is acute otitis media.

73. The method of claim 71, wherein said otitis media is otitis media with effusion.

74. The method of claim 71, wherein said otitis media is chronic otitis media.

75. The method of claim 69, wherein said ear infection is otitis externa.

76. The method of claim 75, wherein said otitis externa is acute otitis externa.

77. The method of claim 75, wherein said otitis extema is chronic otitis externa.

78. The method of claim 75, wherein said otitis externa is malignant otitis extema.

79. The method of claim 69, wherein said composition is administered to the tympanic membrane or external auditory canal of said patient.

80. The method of claim 69, wherein said patient has undergone or will undergo surgery to said ear.

81. The method of claim 80, wherein said composition is administered to the area of the ear to which surgery has been or will be performed.

82. The method of claim 80, wherein said surgery is stapedectomy, tympanoplasty, tympanostomy tube insertion, removal of tumors, or cochlear implant surgery.

83. The method of claim 80, wherein said composition is administered within seven days prior to and after said surgery.

84. The method of claim 69, wherein method further comprises an acidification therapy.

85. The method of claim 84, wherein acidification therapy comprises administering an acetic acid solution to the ear of said patient.

86. The method of claim 69, wherein said microbial infection is Streptococcus spp., Haemophilus influenza, Moraxella catarhalis Staphylococcus intermedius, Pseudomonas spp., Proteus spp., or Escherichia coli.

87. The method of claim 69, wherein said patient is administered one to four drops of said pharmaceutical composition, wherein the rifamycin analogue present in said composition is in an amount between 0.001% and 5% w/v.

88. The method of claim 70, wherein said rifamycin analogue is in the amount between 0.01% and 3% w/v.

89. The method of claim 88, wherein said rifamycin analogue is in the amount between 0.01% and 1% w/v.

90. The method of claim 89, wherein said rifamycin analogue is in the amount between 0.1% and 0.4% w/v.

91. The method of claim 69 wherein said rifamycin analogue is administered for a duration of 1 to 14 days.

92. The method of claim 93, wherein said rifamycin analogue is administered for a duration of 3 to 7 days.

93. The method of claim 69, wherein said method further comprises administering to said patient a second therapeutic agent.

94. The method of claim 93, wherein said second therapeutic agent is an anesthetic.

95. The method of claim 94, wherein said anesthetic is selected from the group consisting of benzocaine, butamben picrate, tetracaine, dibucaine, prilocaine, etidocaine, mepivacaine, bupivicaine, and lidocaine.

96. The method of claim 93, wherein said second therapeutic agent is an anti-inflammatory agent.

97. The method of claim 96, wherein said anti-inflammatory agent is a non-steroidal anti-inflammatory agent.

98. The method of claim 97, wherein said non-steroidal anti-inflammatory agent is selected from the group consisting of detoprofen, diclofenac, diflunisal, etodolac, fenoprofen, flurbiprofen, indomethacin, ketoprofen, mechlofenameate, mefenamic acid, meloxicam, nabumeone, naproxen sodium, oxaprozin, piroxicam, sulindac, tolmeting, celecoxib, rofecoxib, choline salicylate, salsate, sodium salicylate, magnesium salicylate, aspirin, ibuprofen, paracetamol, acetaminophen, and pseudoephedrine.

99. The method of claim 96, wherein said anti-inflammatory agent is a steroid.

100. The method of claim 99, wherein said steroid is selected from the group consisting of hydrocortisone, prednisone, fluprednisolone, triamcinolone, dexamethasone, betamethasone, cortisone, prednisolone, triamcinolone, methylprednisolone, fluocinolone acetonide, flurandrenolone acetonide, and fluorometholone.

101. The method of claim 93 wherein said second therapeutic agent is an antimicrobial agent.

102. The method of claim 101, wherein said antimicrobial agent is selected from the group consisting of amoxillin, azithromycin, clarithromycin, tobramycin, ciprofloxaxin, norfloxacin, gatifloxacin, ofloxacin, levofloxacin, moxifloxacin, metronidazole lomefloxacin, ciprofloxacin, natamycin, neomycin, polymyxin B, gentamycin, bacitracin, trovafloxacin, grepafloxacin, sulfacetamide, tetracycline, gramicidin, chloremphenicol, gramicidin, and erythromycin.

103. The method of claim 93, wherein said second therapeutic agent is a zinc salt.

104. The method of claim 103, wherein said zinc salt is selected from the group consisting of zinc sulfate, zinc chloride, zinc acetate, zinc phenol sulfonate, zinc borate, zinc bromide, zinc nitrate, zinc glycerophosphate, zinc benzoate, zinc carbonate, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, and zinc glycinate.

105. The method of claim 93, wherein said composition and said second therapeutic agent are administered within 24 hours of each other.

106. A method for treating a patient having an infection of Clostridium difficile or preventing an infection of Clostridium difficile in said patient, said method comprising administering to said patient a composition of claim 28 in an amount effective to treat said patient.

107. The method of claim 106, wherein said the rifamycin analogue present in said composition is administered in an amount between 1 and 1000 mg/day.

108. The method of claim 107, wherein said rifamycin analogue is administered in an amount between 1 and 100 mg/day.

109. The method of claim 108, wherein said rifamycin analogue is administered in an amount between 5 and 50 mg/day.

110. The method of claim 109, wherein said rifamycin analogue is administered in an amount between 5 and 25 mg/day.

111. The method of claim 106, wherein said rifamycin analogue is administered for 1 to 14 days.

112. The method of claim 111, wherein said rifamycin analogue is administered for 3 to 7 days.

113. The method of claim 106, wherein said rifamycin analogue is administered as a single dose.

114. The method of claim 106, wherein said rifamycin analogue is administered at an initial dose of between 5 and 100 mg, followed by subsequent doses of between 1 and 50 mg for 3 to 7 days.

115. A pharmaceutical pack comprising (i) a composition of claim 28 in an amount effective to treat a patient having an infection of Clostridium difficile or prevent an infection of Clostridium difficile in said patient; and (ii) instructions for administering said composition to said patient for treating or preventing a Clostridium difficile infection.

116. A method for treating a patient having a sexually transmitted disease caused by an infection of Chlamydia trachomatis or N. gonorrhoeae, said method comprising administering to said patient a single oral dose of a composition of claim 28 in an amount effective to treat said patient.

117. The method of claim 116, wherein the rifamycin analogue present in said composition is administered in an amount between 0.1 and 100 mg.

118. The method of claim 117, wherein said rifamycin analogue is administered in an amount between 1 and 50 mg.

119. The method of claim 118, wherein said rifamycin analogue is administered in an amount between 5 and 25 mg.

120. A method for treating a patient having an infection of C. trachomatis or N. gonorrhoeae, said method comprising administering to said patient a single oral dose of a composition of claim 28 in an amount effective to treat said patient.

121. The method of claim 120, wherein the rifamycin analogue present in said composition is administered in an amount between 0.1 and 100 mg.

122. The method of claim 121, wherein said rifamycin analogue is administered in an amount between 1 and 50 mg.

123. The method of claim 122, wherein said rifamycin analogue is administered in an amount between 5 and 25 mg.

124. A pharmaceutical pack comprising (i) a single oral dose of a composition of claim 28 in an amount effective to treat a patient having a sexually transmitted disease caused by an infection of C. trachomatis or N. gonorrhoeae; and (ii) instructions for administering said single oral dose of said composition to said patient.

125. The pharmaceutical pack of claim 124, wherein said compound is in an amount between 0.1 and 100 mg.

126. The pharmaceutical pack of claim 125, wherein said compound is in an amount between 1 and 50 mg.

127. The pharmaceutical pack of claim 126, wherein said compound is in an amount between 5 and 25 mg.

128. A method of treating a patient having a chronic disease associated with a bacterial infection caused by bacteria capable of establishing a cryptic phase, said method comprising the step of administering to said patient a composition of claim 28 for a time and in an amount sufficient to treat said cryptic phase of said bacterial infection.

129. A method of treating the non-replicating, cryptic phase of a bacterial infection, said method comprising the step of administering to a patient a composition of claim 28 for a time and in an amount sufficient to treat said cryptic phase of said bacterial infection.

130. A method of treating a bacterial infection, said method comprising the steps of:
(a) treating the replicating phase or the elementary body phase of the chlamydial life cycle by administering an antibacterial agent to a patient for a time and in an amount sufficient to treat said replicating phase or elementary body phase of said bacterial infection, and
(b) treating the cryptic phase of the infection by administering to said patient a composition of claim 28 for a time and in an amount sufficient to treat said cryptic phase of said bacterial infection.

131. The method of claim 128, wherein said chronic disease is an inflammatory disease.

132. The method of claim 131, wherein said inflammatory disease is selected from the group consisting of asthma, coronary artery disease, arthritis, conjunctivitis, lymphogranuloma venerum, cervicitis, and salpingitis.

133. The method of claim 128, wherein said chronic disease is an autoimmune disease.

134. The method of claim 133, wherein said autoimmune disease is selected from the group consisting of systemic lupus erythematosus, diabetes mellitus, and graft versus host disease.

135. The method of claim 128, wherein said chronic disease occurs in an immuno-compromised patient.

136. The method of claim 135, wherein said immuno-compromised patient is a patient infected with HIV or a patient undergoing chemotherapy or radiation therapy.

137. The method of claim 128, wherein said bacterial infection is caused by C. trachomatis, C. pneumoniae, C. psittaci, C. suis, C. pecorum, C. abortus, C. caviae, C. felis, C. muridarum, N. hartmannellae, W. chondrophila, S. negevensis, or P. acanthamoeba.

138. The method of claim 128, wherein said patient is administered said composition for at least 30 days.

139. The method of claim 138, wherein said patient is administered said composition for at least 45 days.

140. The method of claim 139, wherein said patient is administered said composition for at least 90 days.

141. The method of claim 140, wherein said patient is administered said composition for at least 180 days.

142. A method for treating a patient having an infection of H. pylori, said method comprising administering to said patient a composition of claim 28 in an amount effective to treat said patient.

143. The method of claim 142, wherein the rifamycin analogue present in said composition is administered in an amount between 1 and 1000 mg/day.

144. The method of claim 143, wherein said rifamycin analogue is administered in an amount between 1 and 100 mg/day.

145. The method of claim 144, wherein said rifamycin analogue is administered in an amount between 5 and 50 mg/day.

146. The method of claim 145, wherein said rifamycin analogue is administered in an amount between 5 and 25 mg/day.

147. The method of claim 142, wherein said rifamycin analogue is administered for 1 to 14 days.

148. The method of claim 147, wherein said rifamycin analogue is administered for 3 to 7 days.

149. The method of claim 142, wherein said rifamycin analogue is administered as a single dose.

150. The method of claim 142, wherein said rifamycin analogue is administered at an initial dose of between 5 and 100 mg, followed by subsequent doses of between 1 and 50 mg for 3 to 7 days.

151. The method of claim 142, further comprising administering to said patient a proton pump inhibitor or bismuth preparation.

152. The method of claim 151, wherein said proton pump inhibitor is selected from the group consisting of omeprazole, lansoprazole, leminoprazole, pantoprazole and robeprazole.

153. The method of claim 151, wherein said bismuth preparation is selected from the group consisting of colloidal bismuth subcitrate and bismuth subsalicylate.

154. A pharmaceutical pack comprising (i) a composition of claim 28 in an amount effective to treat a patient having an infection of H. pylori; and (ii) instructions for administering said composition to said patient for treating or preventing a Clostridium difficile infection.

155. The pharmaceutical pack of claim 154, further comprising a proton pump inhibitor or bismuth preparation.

156. The pharmaceutical pack of claim 155, wherein said proton pump inhibitor is selected from the group consisting of omeprazole, lansoprazole, leminoprazole, pantoprazole and robeprazole.

157. The pharmaceutical pack of claim 155, wherein said bismuth preparation is selected from the group consisting of colloidal bismuth subcitrate and bismuth subsalicylate.
